SEQUENCE LISTING (15 sequences)

SEQ ID NO: 1 - protein, 676 aa, Solanum tuberosum
  PRK-1 (1)..(676)
MAIQKWLLFLFLHLHVLNIVAQLPDLRFGICGKSGNYTENSTYKNDLNTLLTSLSSKIDKYGFYNASIGQNSDRASVIVLCRGDVELDDCRGCVDNVVQKIAQLCPNQKEVFGGYDGCMLQYSNQSIIDTPSLSVQLFLWNTANASKPEEFNQELGKLLENLRDRAAQGGPLQKYATGTTIGPDIQPIYALVQCTPDLSRQSCFDCLTDAYGTLPQCPCLGKTGGRIIGIRCNFRYEISRFFVDVPLEAPPPAGNDNKTVPTGTENKTPPTGKDDKTTRTIIIIVVSTVTIVILMICIAVILIRRRKRKLVNGIQGTSVDDTSIAESFQYDFSAIRAATDDFSDANKLGEGGFGPVYKGKLQNGQEVAVKRLSADSGQGDLESKNEVLLVARLQHRNLVRLLGFCLDGTERLLVYEFVPNASLDHFLFDSVKRRQLDWERRSKIIGGIAKGILYLHEDSRLRIIHRDLKASNVLLDAEMNPKISDFGMARLFELDETQGSTNRIVGTYGYMAPEYAMHGQPSVKSDVFSFGVLVLEILSGQKNTCFRNGESVEDLLSFAWLSWRNGTTIDFVDPMLKESTGLIRDIMRNIHIALLCVQESVADRPTMAAVVLMLSSFSLSLPMPSGPAFYMHSNITAETSLIKEYNTRMTDSSELAKSKSIGSSRNEASISELYPR

SEQ ID NO: 2 - protein, 676 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGCMLQYSNQSILETTSFSLKYYFWNPANATKPGEFNQELGRLLENLRDRAVDDGPLQKYASGNATGPDFQAIYALVQCTPDLSRQSCFSCLSDAYGNMPRCPCLGKRGGRIIGVRCNFRYESSRFFEDVPLEAPPPAGNDNTTVPTGTDDKTVPTGEDDKTTRTIIIIVVSTVTVVILIICIAVILIRRRKRKLVNEIQSTSVDDTSIAESFQYDFSAIRAATDDFSDANKLGEGGFGPVYKGKLQNGQEVAVKRLSADSGQGDLEFKNEVLLVARLQHRNLVRLLGFCLDGTERLLVYEFVPNASLDHFLFDSVKRRQLDWERRSKIIGGIAKGILYLHEDSRLRIIHRDLKASNVLLDAEMNPKISDFGMARLFELDETQGSTNRIVGTYGYMAPEYAMHGQFSVKSDVFSFGVLVLEILSGQKNTCFRNGESVEDLLSFAWSSWRNGTTINFVDPMLKESTGLIRDIMRNIHIALLCVQESVADRPTMAAVVLMLSSFSLSLPMPSGPAFYMHSNITAGTSLIQEYNTRVTDSSERAKSKSIGSSRNEASITELYPR

SEQ ID NO: 3 - protein, 651 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGSNATKPEEFNQELGRLLENLRDRAVDDGPLQKYASGNATGPDFQAIYALVQCTPDLSRQSCFSCLSDAYGNMPRCPCLGKRGGRIIGVRCNFRYESSRFFEDVPLEAPPPAGNDNTTVPTGTDDKTVPTGEDDKTTRTIIIIVVSTVTVVILIICIAVILIRRRKRKLVNEIQSTSVDDTSIAESFQYDFSAIRAATDDFSDANKLGEGGFGPVYKGKLQNGQEVAVKRLSADSGQGDLEFKNEVLLVARLQHRNLVRLLGFCLDGTERLLVYEFVPNASLDHFLFDSVKRRQLDWERRSKIIGGIAKGILYLHEDSRLRIIHRDLKASNVLLDAEMNPKISDFGMARLFELDETQGSTNRIVGTYGYMAPEYAMHGQFSVKSDVFSFGVLVLEILSGQKNTCFRNGESVEDLLSFAWSSWRNGTTINFVDPMLKESTGLIRDIMRNIHIALLCVQESVADRPTMAAVVLMLSSFSLSLPMPSGPAFYMHSNITAGTSLIQEYNTRVTDSSERAKSKSIGSSRNEASITELYPR

SEQ ID NO: 4 - protein, 676 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGCMLQYSNQSILETTSFSLKYYFWNPANATKPEEFNQELGRLLENLRDRAVDDGPLQKYASGNATGPDFQAIYALVQCTPDLSRQSCFSCLSDAYGNMPRRPCLGKRGGRIIGVRCNFRYESSRFFEDVPLEAPPPAGNDNTTVPTGTDDKTVPTGEDDKTTRTIIIIVVSTVTVVILIICIAVILIRRRKRKLVNEIQSTSVDDTSIAESFQYDFSAIRAATDDFSDANKLGEGGFGPVYKGKLQNGQEVAVKRLSADSGQGDLEFKNEVLLVARLQHRNLVRLLGFCLDGTERLLVYEFVPNASLDHFLFDSVKRRQLDWERRSKIIGGIAKGILYLHEDSRLRIIHRDLKASNVLLDAEMNPKISDFGMARLFELDETQGSTNRIVGTYGYMAPEYAMHGQFSVKSDVFSFGVLVLEILSGQKNTCFRNGESVEDLLSFAWSSWRNGTTINFVDPMLKESTGLIRDIMRNIHIALLCVQESVADRPTMAAVVLMLSSFSLSLPMPSGPAFYMHSNITAGTSLIQEYNTRVTDSSERVKSKSIGSSRNEASITELYPR

SEQ ID NO: 5 - DNA, 2227 bp, Solanum tuberousm
atcaaaacttgcttgtaacttaaacagacatagccatggctattcaaaagtggttactctttctgtttttgcatttacatgttctcaatattgtagcgcagctgcctgatttacgattcggtatatgtggtaagagtggtaactatactgagaatagtacctacaagaacgatctaaacacactcctcacttccctttcctcaaaaatagataagtatggtttctacaatgcttctattggccaaaactctgatagagccagcgttattgtgttatgtagaggagatgtggagctagacgattgtcgcggttgtgtcgataatgttgttcaaaagattgcacagttatgtcctaaccaaaaagaagttttcggtggctatgatggatgtatgttacagtattcgaatcaatccattatagatactccatcgttgtctgttcaattatttctttggaatactgcgaatgcctcaaaacccgaggaatttaaccaggagctaggaaaattattggaaaatttacgagaccgtgctgcacagggtggtcctcttcaaaaatatgctactggtactacgataggtccagatattcagcctatatatgcacttgtgcagtgtactcctgatttatctcgtcagagttgcttcgattgcttaactgacgcttatggaaccttgcctcaatgtccctgcctgggaaagaccggtggaagaatcatagggattaggtgcaacttccgttatgaaatttcccgtttcttcgtcgatgtgccgttggaagctccgccacctgcaggaaatgataataaaacagttccaacagggacggaaaataaaacacctccaactgggaaggacgataaaacaacacgaacaattatcattatcgttgtgtcaactgttacaattgttattcttatgatttgtattgctgtcatcttgataaggaggcgaaagaggaaactggtgaacggaattcagggtacatctgtagatgatactagtattgcagaatcttttcaatatgatttttcggcaattagagcagcaacagatgacttctcagatgctaataagcttggagaaggtggatttggtcctgtgtacaagggtaagcttcaaaatggacaagaagtagcagtgaaaaggttatcagcagattcaggccaaggtgatctagaatccaaaaatgaggtcttgttggttgccaggcttcaacacaggaatttggttaggttgctgggtttttgcctagacggaacagagcgacttcttgtctatgagtttgttcccaatgcaagtcttgaccacttcttatttgattcagttaaacgtaggcaattggattgggaaaggcgatccaaaatcataggaggcattgctaagggaattctttatcttcatgaggattctaggcttcggatcattcaccgtgatctcaaagctagtaatgttctactagatgcagaaatgaatcctaaaatctcagattttggcatggcaaggctatttgaattagatgaaactcaaggcagcacaaacagaattgttgggacctatggatatatggcaccagagtatgcaatgcacgggcaattttccgtaaagtcagatgtttttagctttggagtactagtcttagaaattttaagtgccaaaaaaaacacttgtttcagaaatggagaatcggtggaagaccttttgagttttgcttggttaagctggcgtaatggaacaactatagattttgtagatccaatgctgaaggaaagcacaggactgattcgtgacataatgagaaacattcacatagctttattgtgcgttcaagaaagtgtggctgatagaccaaccatggcagctgttgttctcatgctcagtagcttttcgttgagtcttccaatgccttcagggccagcattctatatgcacagtaatattaccgcagagacgtcgcttattaaagaatacaacacaagaatgacagactctagtgagctagccaaaagtaaatctattggttcatcacgaaatgaggcatccatatctgagttatatcctcgttaacatccctagtatgataacctttttctctgagtttacttattttatgtgttggtaccacttttagtgtacgtgtagtctagctaacaataattattataaagtttatatgtaatagtaacactctttttaatacagaaaaaaaaaaaaaaaaaaaaaaaaaa

SEQ ID NO: 6 - DNA, 2242 bp, Solanum tuberosum
atcaaaacttgcttgtaacttaaacagacatagccatggctattcaaaagtggctactcattctgtttttgcatatacatgttctcaatattgtagcgcagctgcctgatttacgatttggttcatgtggtggcaatggtaactatactgagaatagtacctacaagaacaatctaaacacactcctcacttccctttcctcaaaaatagacaactatggtttctacaatgcttctattggtcaaaactctgatagagccagcgttattgtgttatgtagaggagatgtggagctagccgattgtcgcggttgtgtcgataatgttgttcaaaagattgcacagttatgtcctaaccaaaaagaagttttcggtggctatgatggatgtatgttacagtattcaaatcaatccattttagaaactacatcattttctcttaaatattacttttggaatccagcgaatgccacaaaacccggggaatttaaccaggagctagggagattattgggaaacttacgagatcgtgctgtagatgatggtcctcttcaaaaatatgctagtggtaatgcgacaggtccagattttcaggctatatatgcacttgtgcagtgcactcctgatttatctcgtcagagttgcttcagttgcttaagtgacgcttatggaaacatgcctcgctgtccctgcctgggtaagaggggtggaagaatcataggggttaggtgcaacttccgttatgaaagttcccgtttcttcgaggatgtgccgttggaagctccaccacctgcaggaaatgataatacaacagttccaacagggacggacgataaaacagttccaacaggagaggatgacaaaacaacacgaacaatcatcattatcgttgtgtcaactgttacagttgttattcttattatttgtattgctgtcatcttgataaggaggcgaaagaggaagctggtgaacgaaattcagagtacatctgtagatgatactagtattgcagaatcttttcaatatgatttttcggcaattagagcagcaacagatgacttctcagatgctaataagctcggagaaggcggatttggtcctgtgtacaagggtaagcttcaaaatggacaagaagtagcagtgaaaaggttatcagcagattcaggccaaggtgatctagaattcaaaaatgaggtcttgttggttgccaggcttcaacacaggaatttggttaggttgctgggattttgcctagacggaacagagcgacttcttgtctatgagtttgttcccaatgcaagtcttgaccacttcttatttgattcagttaaacgtaggcaattggattgggaaaggcgatccaaaatcataggaggcattgctaagggaattctttatcttcatgaggattctaggcttcggatcattcaccgtgatctcaaagctagtaatgttctactagatgcagaaatgaatcctaaaatctcagattttggcatggcaaggctatttgaattagatgaaactcaaggcagcacaaacagaattgttgggacctatggatatatggcaccagaatatgcaatgcacgggcaattttctgtgaagtcagatgtttttagctttggagtactagtcttagaaattttaagtggccaaaaaaacacttgtttcagaaatggagaatcggtggaagaccttctgagttttgcttggtcgagctggcgtaatggaacaactataaattttgtagatccaatgctgaaggaaagcacaggactgattcgtgacataatgagaaacattcacatagctttattgtgtgttcaagaaagtgtggctgatagaccaaccatggcagctgttgttctcatgctcagtagcttttcgttgagtcttcccatgccttcagggccagcattctatatgcacagtaatattaccgcagggacgtcgcttattcaagaatacaacacaagagtgacagactctagtgaacgagccaaaagtaaatctattggttcatcacgaaatgaggcgtccataactgagttatatcctcgttaacatctttagtataatagcctttttctccgattttacttattttacctgccttggacttgtagtctagctagcaattattgtaaagttttatttaacttgagctaaatacatctatatccattttaactggcttgctttgtaagatagcatcatttcgccaaaaaaaaaaaaaaaaa

SEQ ID NO: 7 - DNA, 2144 bp, Solamum tuberosum
atcaaaacttgcttgtaacttaaacagacatagccatggctattcaaaagtggctactcattctgtttttgcatatacatgttctcaatattgtagcgcagctgcctgatttacgatttggttcatgtggtggcaatggtaactatactgagaatagtacctacaagaacaatctaaacacactcctcacttccctttcctcaaaaatagacaactatggtttctacaatgcttctattggtcaaaactctgatagagccagcgttattgtgttatgtagaggagatgtggagctagccgattgtcgcggttgtgtcgataatgttgttcaaaagattgcacagttatgtcctaaccaaaaagaagttttcggtggctatgatggatcgaatgccacaaaacccgaggaatttaaccaggagctagggagattattggaaaacttacgagatcgtgctgtagatgatggtcctcttcaaaaatatgctagtggtaatgcgacaggtccagattttcaggctatatatgcacttgtgcagtgtactcctgatttatctcgtcagagttgcttcagttgcttaagtgacgcttatggaaacatgcctcgctgtccctgcctgggtaagaggggtggaagaatcataggggttaggtgcaacttccgttatgaaagttcccgtttcttcgaggatgtgccgttggaagctccaccacctgcaggaaatgataatacaacagttccaacagggacggacgataaaacagttccaacaggagaggatgacaaaacaacacgaacaatcatcattatcgttgtgtcaactgttacagttgttattcttattatttgtattgctgtcatcttgataaggaggcgaaagaggaagctggtgaacgaaattcagagtacatctgtagatgatactagtattgcagaatcttttcaatatgatttttcggcaattagagcagcaacagatgacttctcagatgctaataagctcggagaaggcggatttggtcctgtgtacaagggtaagcttcaaaatggacaagaagtagcagtgaaaaggttatcagcagattcaggccaaggtgatctagaattcaaaaatgaggtcttgttggttgccaggcttcaacacaggaatttggttaggttgctgggattttgcctagacggaacagagcgacttcttgtctatgagtttgttcccaatgcaagtcttgaccacttcttatttgattcagttaaacgtaggcaattggattgggaaaggcgatccaaaatcataggaggcattgctaagggaattctttatcttcatgaggattctaggcttcggatcattcaccgtgatctcaaagctagtaatgttctactagatgcagaaatgaatcctaaaatctcagattttggcatggcaaggctatttgaattagatgaaactcaaggcagcacaaacagaattgttgggacctatggatatatggcaccagaatatgcaatgcacgggcaattttctgtgaagtcagatgtttttagctttggagtactagtcttagaaattttaagtggccaaaaaaacacttgtttcagaaatggagaatcggtggaagaccttctgagttttgcttggtcgagctggcgtaatggaacaactataaattttgtagatccaatgctgaaggaaagcacaggactgattcgtgacataatgagaaacattcacatagctttattgtgtgttcaagaaagtgtggctgatagaccaaccatggcagctgttgttctcatgctcagtagcttttcgttgagtcttcccatgccttcagggccagcattctatatgcacagtaatattaccgcagggacgtcgcttattcaagaatacaacacaagagtgacagactctagtgaacgagccaaaagtaaatctattggttcatcacgaaatgaggcgtccataactgagttatatcctcgttaacatctttagtataatagcctttttctccgattttacttattttacctgccttggacttgtagtctagctagcaattattgtaaagttttatttaacttgagctaaatacatctatatccattccaaaaaaaaaaaaaaaaaaaaaaaaaaaaa

SEQ ID NO: 8 - DNA, 2411 bp, Solanum tuberosum
aaaacttgcttgtaacttaaacagacatagccatggctattcaaaagtggctactcattctgtttttgcatatacatgttctcaatattgtagcgcagctgcctgatttacgatttggttcatgtggtggcaatggtaactatactgagaatagtacctacaagaacaatctaaacacactcctcacttccctttcctcaaaaatagacaactatggtttctacaatgcttctattggtcaaaactctgatagagccagcgttattgtgttatgtagaggagatgtggagctagccgattgtcgcggttgtgtcgataatgttgttcaaaagattgcacagttatgtcctaaccaaaaagaagttttcggtggctatgatggatgtatgttacagtattcaaatcaatccattttagaaactacatcattttctcttaaatattacttttggaatccagcgaatgccacaaaacccgaggaatttaaccaggagctagggagattattggaaaacttacgagatcgtgctgtagatgatggtcctcttcaaaaatatgctagtggtaatgcgacaggtccagattttcaggctatatatgcacttgtgcagtgtactcctgatttatctcgtcagagttgcttcagttgcttaagtgacgcttatggaaacatgcctcgccgtccctgcctgggtaagaggggtggaagaatcataggggttaggtgcaacttccgttatgaaagttcccgtttcttcgaggatgtgccgttggaagctccaccacctgcaggaaatgataatacaacagttccaacagggacggacgataaaacagttccaacaggagaggatgacaaaacaacacgaacaatcatcattatcgttgtgtcaactgttacagttgttattcttattatttgtattgctgtcatcttgataaggaggcgaaagaggaagctggtgaacgaaattcagagtacatctgtagatgatactagtattgcagaatcttttcaatatgatttttcggcaattagagcagcaacagatgacttctcagatgctaataagctcggagaaggcggatttggtcctgtgtacaagggtaagcttcaaaatggacaagaagtagcagtgaaaaggttatcagcagattcaggccaaggtgatctagaattcaaaaatgaggtcttgttggttgccaggcttcaacacaggaatttggttaggttgctgggattttgcctagacggaacagagcgacttcttgtctatgagtttgttcccaatgcaagtcttgaccacttcttatttgattcggttaaacgtaggcaattggattgggaaaggcgatccaaaatcataggaggcattgctaagggaattctttatcttcatgaggattctaggcttcggatcattcaccgtgatctcaaagctagtaatgttctactagatgcagaaatgaatcctaaaatctcagattttggcatggcaaggctatttgaattagatgaaactcaaggcagcacaaacagaattgttgggacctatggatatatggcaccagaatatgcaatgcacgggcaattttctgtgaagtcagatgtttttagctttggagtactagtcttagaaattttaagtggccaaaaaaacacttgtttcagaaatggagaatcggtggaagaccttctgagttttgcttggtcgagcgtgcgtaatggaacaactataaattttgtagatccaatgctgaaggaaagcacaggatgattcgtgacataatgagaaaacattcacatagctttattgtgtgttcaagaaagtgtggctgatagaccaaccatggcagctgttgttctcatgctcagtagcttttcgttgagtcttcccatgccttcagggccagcattctatatgcacagtaatattaccgcagggacgtcgcttattcaagaatacaacacaagagtgacagactctagtgaacgagtcaaaagtaaatctattggttcatcacgaaatgaggcgtccataactgagttatatcctcgttaacatctttagaataatagcctttttctccgattttacttattttacctgccttggacttgtagtctagctagcaattattgtaaagttttatttaacttgagctaaatacatctatatccattttaactggcttgctttgtaagatagcatcatttcggcatctcacacgtcttttcaactttatggaagaggcaaaagggaaaagaaaacaagaggaaaggaaaaggtctcaatttctagaactccttccgtacccctctttctttatgttattgaagagccaaataacttttcgtattctaaattcatataaagttttactccaaaaaaaaaaaaaaaaaaaaaaaa

SEQ ID NO: 9 - DNA, 1975 bp, Arabidopsis thaliana
atgtcttattactcctctttcttcttccttttcctcttctcctttctcacgagtttcagagtttctgctcaagatcccacttacgtataccacacctgtcaaaatacggcaaattacacaagtaacagcacttacaacaacaatctcaagacccttttggcttctctttcttcccgcaacgcttcccactccaccggattccaaaacgccaccgtcggccaagctccagacagagtcaccggacttttcaactgccgtggagatgtctcaacggaagtttgccgtagatgcgtctcctttgccgtcaacgacaccttaactcggtgtcctaaccagaaagaagccacactttattacgatgagtgtgtacttagatactctaaccagaatattctctcgacactaataaccaccggtggagttatctggttaacaccccggaatgtgacatctaaccaactagacctgttaagtgatttggttttgcctactctgaaccaagccgccaccgtagccttgaacagttccaaaaattcggtacgagaaaaaacaattttactgcgttgcagagtttctatggactggttcagtgcacacctgatctcacgagacaagactgttcgcgttgtctccaactggtcatcaatcaaatacctactgacagaattggtgcaagaattattaatccgagctgtacttcaagatacgagatctacgcgttttacaccgaatccgccgttccaccaccaccaccaccaccgtcaatttctactcctccagtttcagctcctccgcgatctggaaaagatgggaattcaaaggtgttagtgatagccattgttgtgcctattatagtggctgttctgctttttatagctggttattgtttccttacaagaagggcaaggaagagttattacacaccatctgcatttgctggagatgatatcacaacagcagactcactacagcttgattatagaacaattcaaactgcaacagatgattttgtagagagtaataagattggtcaaggtggatttggtgaggtttacaagggtactttatcggatgggactgaagttgcggtgaagcgactatcaaagtcatcaggacaaggtgaagtagagttcaagaacgaggttgttcttgttgcaaagctacaacatagaaatctggttagacttcttgggttttgtctagacggagaagaaagagtactggtctacgagtatgtgcccaacaaaagtcttgattacttcctctttgaccctgcaaagaaaggccagctggattggactcgacgatacaagattatcggtggagttgctcgagggattctgtatcttcatcaagactgacgactcacaatcatacaccgtgacctcaaagccagtaacattctcttggacgcggatatgaatcctaaaattgcagattttggaatggctaggatctttggattggaccaaaccgaagagaacacaagcagaatagttggtacctatggttacatgtctcccgagtatgcaatgcacggtcagtactcaatgaaatctgatgtctatagcttcggagtgttagttcttgagattataagtggcaagaagaatagcagcttctaccaaacagatggtgcacatgacttggtctcatatgcttgggggctttggagtaacgggcgaccattagaactcgtggatccagctattgtagaaaattgccaaaggaatgaagttgttcgatgtgtccatatcggtcttttatgtgttcaagaagatcctgctgagcgtccgacgttgtcaaccattgttctgatgctcactagtaacactgtgactttacccgtgccatggcaacccggtcttttctttcagagtagaattggaaaagacccgcttgatacagatacaacgagcaaatctcttctagggtctgttgacgatgcatccatcacagatatacatcctcgatg

SEQ ID NO: 10 - protein, 278 aa, Solanum tuberosum
MAIQKWLLFLFLHLHVLNIVAQLPDLRFGICGKSGNYTENSTYKNDLNTLLTSLSSKIDKYGFYNASIGQNSDRASVIVLCRGDVELDDCRGCVDNVVQKIAQLCPNQKEVFGGYDGCMLQYSNQSIIDTPSLSVQLFLWNTANASKPEEFNQELGKLLENLRDRAAQGGPLQKYATGTTIGPDIQPIYALVQCTPDLSRQSCFDCLTDAYGTLPQCPCLGKTGGRIIGIRCNFRYEISRFFVDVPLEAPPPAGNDNKTVPTGTENKTPPTGKDDKTT

SEQ ID NO: 11 - protein, 278 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGCMLQYSNQSILETTSFSLKYYFWNPANATKPGEFNQELGRLLENLRDRAVDDGPLQKYASGNATGPDFQAIYALVQCTPDLSRQSCFSCLSDAYGNMPRCPCLGKRGGRIIGVRCNFRYESSRFFEDVPLEAPPPAGNDNTTVPTGTDDKTVPTGEDDKTT

SEQ ID NO: 12 - protein, 253 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGSNATKPEEFNQELGRLLENLRDRAVDDGPLQKYASGNATGPDFQAIYALVQCTPDLSRQSCFSCLSDAYGNMPRCPCLGKRGGRIIGVRCNFRYESSRFFEDVPLEAPPPAGNDNTTVPTGTDDKTVPTGEDDKTT

SEQ ID NO: 13 - protein, 278 aa, Solanum tuberosum
MAIQKWLLILFLHIHVLNIVAQLPDLRFGSCGGNGNYTENSTYKNNLNTLLTSLSSKIDNYGFYNASIGQNSDRASVIVLCRGDVELADCRGCVDNVVQKIAQLCPNQKEVFGGYDGCMLQYSNQSILETTSFSLKYYFWNPANATKPEEFNQELGRLLENLRDRAVDDGPLQKYASGNATGPDFQAIYALVQCTPDLSRQSCFSCLSDAYGNMPRRPCLGKRGGRIIGVRCNFRYESSRFFEDVPLEAPPPAGNDNTTVPTGTDDKTVPTGEDDKTT

SEQ ID NO: 14 - protein, 658 aa, Arabidopsis thaliana
MSYYSSFFFLFLFSFLTSFRVSAQDPTYVYHTCQNTANYTSNSTYNNNLKTLLASLSSRNASHSTGFQNATVGQAPDRVTGLFNCRGDVSTEVCRRCVSFAVNDTLTRCPNQKEATLYYDECVLRYSNQNILSTLITTGGVILVNTRNVTSNQLDLLSDLVLPTLNQAATVALNSSKKFGTRKNNFTALQSFYGLVQCTPDLTRQDCSRCLQLVINQIPTDRIGARIINPSCTSRYEIYAFYTESAVPPPPPPPSISTPPVSAPPRSGKDGNSKVLVIAIVVPIIVAVLLFIAGYCFLTRRARKSYYTPSAFAGDDITTADSLQLDYRTIQTATDDFVESNKIGQGGFGEVYKGTLSDGTEVAVKRLSKSSGQGEVEFKNEVVLVAKLQHRNLVRLLGFCLDGEERVLVYEYVPNKSLDYFLFDPAKKGQLDWTRRYKIIGGVARGILYLHQDSRLTIIHRDLKASNILLDADMNPKIADFGMARIFGLDQTEENTSRIVGTYGYMSPEYAMHGQYSMKSDVYSFGVLVLEIISGKKNSSFYQTDGAHDLVSYAWGLWSNGRPLELVDPAIVENCQRNEVVRCVHIGLLCVQEDPAERPTLSTIVLMLTSNTVTLPVPWQPGLFFQSRIGKDPLDTDTTSKSLLGSVDDASITDIHPR

SEQ ID NO: 15 - DNA, 877 bp, Solanum tuberosum
gagaggatctcaaagaaacagtatgcatttttcccaatttatgtttgctgttaatgtaacctatgaatgatttagacaattgtaaattatctacctaagttgtagtcttttatgttgtgtaccctttgatgaagttctgtgatttttgtcttttggttactaaggggttgttgtttttgtcttccacttttaatatggtttatttcttgaagttaaagattggatttttatgtcacttgtaaaaaaggggaataaaaagattggatttttatgtctgttctgactgagttgacttcaaacacttgaattggttactaaagggtgttgtttttgtcttccacttttaatctggtctatttcttgaagtaaaaaagattgaatttttatgtcacttacaaaaaaaggaacaaaaagattggatttttatgtctgttctgactgagtgaactacaaacatttgattctgaggtgtgtgtaatacttagagatggtatcgatataatggtattcatatgcttcagtatagtttgactgtgttagtggatgaaatcccatgtgtatgttgacttgagatcctaaacttgtgttgaccaagtagtcttgttttttcggaatgttttgtcgtgttttctttagtatttgccagatttcttcacttctgttatttctttttcgatcttattttccttaagctgagggtatatcagaaacagtctctctacctctcaaggtaggggtaaggtcttggtacactctaccctccccagaccctacttgtggaattacactgagtatgttgttgtaattgcaaaagactggtcaacatttaagtctttgatattttcaatctcacattaggtccaagagtgttatgagc

(19) United States
(12) Patent Application Publication
(10) Pub. No.: US 2006/0162019 A1
(43) Pub. Date: Jul. 20, 2006

(54) Novel plant receptor-like kinases

(76) Inventors: Tapio Palva, Porvoo (FI); Marcos Montesano, Montevideo (UY)

Correspondence Address: John Dodds, 1707 N St. NW, Washington, DC, 20036, US

(21) Appl. No.: 11/326,946

(22) Filed: Jan. 6, 2006

Related U.S. Application Data
(63) Continuation-in-part of application No. 10/304,946, filed on Nov. 27, 2002, now abandoned.

(51) Int. Cl. A01H 1/00; C07H 21/04; C12N 9/12; C12N 15/82; A01H 5/00; C12N 5/04

(52) U.S. Cl. 800/279; 800/317.2; 435/194; 435/419; 435/468; 536/23.2

(57) ABSTRACT

The present invention is related to plant molecular biology. Particularly it is related to nucleic acids and methods for conferring disease resistance in plants. It is also related to potato receptor-like kinases (PRKs) and PRK cDNA or nucleic acid sequences and their gene products for conferring enhanced resistance to pathogens and pests. The invention is further related to novel receptors and ligands and their use in detecting plant-pathogen interactions.

### PRIORITY

This application is a Continuation in Part application of the U.S. patent application Ser. No. 10/304,946 which is incorporated herein by reference. This application claims priority of the Finnish patent application number 20012419 also incorporated herein by reference.

### SEQUENCE DATA

This application contains sequence data provided on a computer readable diskette and as a paper version. The paper version of the sequence data is identical to the data provided on the diskette.

### TECHNICAL FIELD OF THE INVENTION

The present invention is related to plant molecular biology. Particularly it is related to nucleic acids and methods for conferring disease resistance in plants. It is also related to potato receptor-like kinases (PRKs) and PRK cDNA or nucleic acid sequences and their gene products as well as fragments and derivatives thereof useful in conferring enhanced resistance to pathogens and pests. The invention is further related to novel receptors and ligands and their use in detecting plant-pathogen interactions.

### BACKGROUND OF THE INVENTION

Potato is the 4^{th} major food crop of the world and expanding. Potato's susceptibility to pests and diseases makes the crop the number two user of agricultural pesticides worldwide, following cotton. Erwinia carotovora is the etiological agent of soft rot disease and can attack a wide range of economically important crops including potato (Pérombelon and Kelman, 1980). The production of extracellular plant cell wall-degrading enzymes, including cellulases, pectinases and proteases is central to virulence of E. carotovora. These enzymes both produce the maceration symptoms in infected plant tissues and release nutrients for bacterial growth (Collmer and Keen, 1986; Kotoujansky 1987; Pirhonen et al., 1991). Many of the plant cell wall-degrading enzymes have been shown to trigger plant defense responses, probably by releasing cell wall fragments active as elicitors (Davis and Ausubel, 1989; Davis et al., 1984; Palva et al., 1993; Vidal et al., 1997, 1998). It has been previously demonstrated that cell-free culture filtrates (CF) containing the cell wall-degrading enzymes of E. carotovora subsp. carotovora, as well as preparations containing single enzymes, induce several pathogenesis-related genes in plants (Norman et al., 1999, Norman-Setterblad et al., 2000; Palva et al., 1993; Vidal et al., 1997, 1998). In addition, it has been shown that several of these defense-related genes are also responsive to oligogalacturonides (Norman et al., 1999).

Plant receptor-like kinases (RLKs) are proteins with a predicted signal sequence, single transmembrane region, and cytoplasmic kinase domain. Plant RLKs show serine/threonine kinase specificity. Based on the structure of the putative extracellular domains, plant receptor-like kinases (RLKs) have been classified into several major classes (Braun and Walker, 1996; Walker, 1994). These include (i) the S-domain RLKs which contain extracellular domains homologous to the S-locus glycoproteins of Brassicaceae (Nasrallah et al., 1993; Walker and Zhang, 1990, Stein et al., 1991), (ii) the leucine rich repeat (LRR) RLKs such as Xa21 from rice, TMK1 and RLK5 from Arabidopsis (Chang et al., 1992; Song et al., 1995; Walker, 1993) and (iii) RLKs with the epidermal growth factor-like repeat (EGF) such as pro25 and the WAKs from Arabidopsis (He et al., 1999; Kohorn et al., 1992). Moreover, several RLKs with different types of extracellular domains have been identified recently (reviewed by Satterlee and Sussman, 1998).

The expression of plant RLK genes have shown diverse patterns, while some of them have displayed expression only in vegetative tissues (Kohorn et al., 1992), others were expressed only in reproductive tissues (Goring et al., 1992; Stein et al., 1991) and some have been shown in both vegetative and reproductive tissues (Pastuglia et al., 1997). Some of the RLK genes are responsive to pathogens and elicitors, including PvRK20-1 from Phaseolus vulgaris (Lange et al., 1999), SFR2 from Brassica olearacea (Pastuglia et al., 1997), Wak1 (He et al., 1998) and RLKs (Du and Chen, 2000) from Arabidopsis thaliana and the disease resistance gene Xa21 from rice (Song et al., 1995).

In plants very little is known about the nature of the ligands interacting with serine-threonine RLKs. Recently, it has been shown that the extracellular domain of a RLK from Arabidopsis, BRI1, perceives brassinosteroids (He et al., 2000). On the other hand, in animals it has been shown that the interleukin 2 and the epidermal growth factor receptors are up regulated by their own ligands (Clark et al., 1985; Deeper et al., 1985).

In order to fully understand the mechanisms of plant disease resistance and provide plants with enhanced disease resistance to pathogens and even to herbivores (insects pests) novel means for studying the interaction between ligand and receptor in plant-pathogen interaction are needed.

The present disclosure provides a solution to said problem. Novel potato receptor-like kinases (PRKs) as well as a new expression pattern are described: PRK is induced by the potato pathogen Erwinia carotovora as well as short oligouronides, which may be the elicitors released by Erwinia. Oligouronides may constitute the ligands for the novel receptor. Oligouronide receptor has not been described previously. The structural identity of PRKs and their induction pattern suggested that they constitute part of the early response of potato E. carotovora infection.

One embodiment of the present invention is to provide isolated nucleic acid sequences comprising polynucleotides encoding receptor-like protein kinases, which comprise preferably in the extracellular domain one or more cystein repeats, characterized in that potato receptor-like kinase (PRK)-like nucleic acid sequences are capable of encoding potato receptor-like kinases (PRKs) substantially homologous to gene products of PRK (SEQ ID NO: 1, SEQ ID NO: 2, SEQ ID NO: 3 or SEQ ID NO: 4) or fragments or derivatives thereof and having substantially the same properties or functions as SEQ ID NO: 1, SEQ ID NO: 2, SEQ ID NO: 3 or SEQ ID NO: 4.

Another embodiment of the present invention is to provide new methods for conferring resistance to pathogens and herbivores (insect pests) producing/releasing elicitors of the present invention.

Still another embodiment of the present invention is to provide new means and methods for making it possible to initiate the defense mechanisms during the early stages of the plant-pathogen interaction.

A further embodiment of the present invention is to provide means carrying out said methods in form of potato receptor-like kinase (PRK) cDNA and PRK nucleic acid sequences, fragments and derivatives thereof as well as their complementary strands and PRK gene products expressed by the PRK nucleic acid sequences of the present disclosure.

Another embodiment of the present invention is to use PRK gene products, fragments and derivatives thereof for technically modifying the expression of a gene or a modified gene/a natural variant of the gene to sensitize the plant to pathogen perception and to generate enhanced resistance.

Still another embodiment of the present invention is to use the ligand of the receptor of the present invention for spraying, inoculating, spreading, applying or by other means the crop plants to enhance disease resistance.

Another embodiment is to use PRK-like nucleic acid sequences as well as their expression products for manufacturing transgenic plant cells or plants with enhanced pathogen resistance.

Another embodiment is to use PRK-like nucleic acid sequences and PRK-like gene products and derivatives and fragments thereof as growth regulators, for induction of development, for detecting different stress conditions. Different stress conditions can be caused e.g. by pathogens and pests, mechanical, chemical or physical stress.

### SUMMARY OF THE INVENTION

The present invention is related to potato receptor-like kinases (PRKs) and PRK-like nucleic acid sequences and fragments and derivatives thereof and their products useful in conferring enhanced resistance to pathogens and pests.

The present invention provides four isolated nucleic acid sequences comprising SEQ ID NO: 5, SEQ ID NO: 6, SEQ ID NO: 7, SEQ ID NO: 8.

Said nucleic acid sequences comprise genes encoding novel potato receptor-like kinases (PRKs) (PRK-1, PRK-2, PRK-3 and PRK-4), SEQ ID NO: 1, SEQ ID NO: 2, SEQ ID NO: 3, SEQ ID NO: 4. They are proposed to belong to a new class of receptor-like protein kinases. Potato PRKs are 41-55% i.e. highly distinct in the extracellular domain from other receptor-like protein kinases of the same class, PvRK20-1 from Phaseolus vulgaris (Lange et al., 1999) and genes from the genome project of Arabidopsis thaliana (Accession numbers CAB38617, CAB81062, CAA18704).

A new expression pattern is described: PRK is induced by the potato pathogen Erwinia carotovora as well as short oligouronides, which may be the elicitors released by Erwinia. Oligouronides may constitute the ligands for the receptor. Oligouronide receptor has not been described previously.

The present invention is related to isolated nucleic acid sequences comprising polynucleotides encoding receptor-like protein kinases, which comprise preferably in the extracellular domain one or more cystein repeats, characterized in that potato receptor-like kinase (PRK)-like nucleic acid sequences are capable of encoding potato receptor-like kinases (PRKs) substantially homologous to gene products of PRK (SEQ ID NO:1, SEQ ID NO:2, SEQ ID NO:3 or SEQ ID NO:4) or fragments or derivatives thereof and having substantially the same properties or functions as SEQ ID NO:1, SEQ ID NO:2, SEQ ID NO:3 or SEQ ID NO:4. The potato receptor-like kinase (PRK)-like nucleic acid sequences are capable of hybridizing with SEQ ID NO:5, SEQ ID NO:6, SEQ ID NO:7 or SEQ ID NO:8 or complementary strands thereof under defined conditions.

The nucleic acid sequences comprise SEQ ID NO: 1, SEQ ID NO: 2, SEQ ID NO: 3 or SEQ ID NO: 4 obtainable from potato under defined conditions. The extracellular domains comprise a conserved bi-modular pattern of one or more cysteine repeats. The expression of genes encoding for gene products SEQ ID NO: 1, SEQ ID NO: 2, SEQ ID NO: 3 or SEQ ID NO: 4 is induced by Erwinia carotovora.

The expression of genes encoding for gene products SEQ ID NO: 1, SEQ ID NO: 2, SEQ ID NO: 3 or SEQ ID NO: 4 is induced by oligouronides. Potato receptor-like kinases (PRKs) function as receptors for ligands released during plant stress conditions by pathogens. Potato receptor-like kinases (PRKs) are formed by alternative splicing. Potato receptor-like kinases (PRKs) are involved in signal perception during potato defense responses against Erwinia carotovora. The expression of potato receptor-like kinases (PRKs) is incuced by response to elicitors, which can be oligouronides or oligogalacturonides.

The PRK-like gene products are polypeptides comprising in their extracellular domains a conserved bi-modular pattern of one or more cysteine repeats. The potato receptor-like kinase (PRK)-like gene products comprise polypeptides having amino acid sequences substantially homologous with SEQ ID NO: 1, SEQ ID NO: 2, SEQ ID NO: 3 or SEQ ID NO: 4. The potato receptor-like kinase (PRK)-like gene products are polypeptides substantially similar to the gene products encoded by SEQ ID NO: 5, SEQ ID NO: 6, SEQ ID NO: 7 or SEQ ID NO: 8.

The present invention is related to the method for preventing plant diseases or enhancing disease resistance to pathogens or herbivores by transforming a plant with a DNA construct comprising nucleic acid sequences encoding potato-receptor like kinase (PRK)-like gene products or fragments thereof functionally combined with regulatory sequences. The present invention is also related to DNA constructs, expression vectors and host cells comprising the DNA sequences of the present invention.

The present invention is related to a method for conferring resistance to pathogens in a plant, preferably potato, so that the method comprises introducing into the plant a recombinant expression construct comprising a plant promoter operably linked to a potato receptor-like kinase (PRK)-like nucleotide sequence or derivatives or fragments thereof encoding a potato receptor-like kinase (PRK)-like gene product.

Other features, aspects and advantages of the present invention will become apparent from the following description and appended claims.

### SHORT DESCRIPTION OF THE FIGURES

FIG. 1 depicts the comparison of the deduced amino acid sequences of PRKs (SEQ ID NO: 1, SEQ ID NO:2, SEQ ID NO:3 and SEQ ID NO:4). Identical amino acids are highlighted with black and similar amino acids with gray. Dashes indicate gaps introduced to improve the alignment. The two hydrophobic regions flanking the putative extracellular domain are double underlined and asterisks indicate the region of basic residues. The numbered brackets indicate putative glycosylation sites. Note that under the brackets number 4 and 6 the putative glycosylation sites are missing from PRK-3 and PRK-1, respectively. The cDNA clones corresponding to the PRKs were sequenced at the DNA Synthesis and Sequencing Unit of the Institute of Biotechnology, Helsinki, Finland, using ABI 377 system. The alignment was performed using PILE UP from the Genetics Computer Group (GCG) software package.

FIG. 2 depicts the structural analysis of PRKs. (a) Comparison of the kinase domains of PRK-1, 2, 3 and 4 with the kinase domain of SFR2 (Pastuglia et al., 1997; accession number P93068), IRK1 (Kowyama et al., 1996; accession number Q40096), PvRK20-1 (Lange et al., 1999; accession number AF078082) and a putative Arabidopsis thaliana (At) receptor-like kinase (Bevan et al., Unpublished data; accession number 065470). The 11 characteristic subdomains of kinases are indicated by roman numbers, and the 15 invariant amino acids are indicated by asterisks and highlighted with black. The two regions in subdomains VI and VIII indicative of serine-threonine kinases are boxed and shaded in gray. Consensus indicates the conserved residues in all sequences shown. (b) Alignment of the extracellular domains of PRK-2, 4 and 3 at the region where PRK-3 lacks 25 amino acids. The cDNA and the translated amino acid sequences near the possible splice sites are shown and the 25 amino acids present in PRK-2 and 4 but not in 3 are highlighted in black. Conserved sequences of splice sites (Brown and Simpson, 1998) are highlighted in gray. An asterisk indicates a conserved cysteine and the bracket indicates a putative N-glycosylation site. The nucleotides generating a different codon in PRK-3 are underlined, as is the amino acid that is altered as a result of the splicing event. (c) Alignment of the extracellular domains of PRK-1, 2, 3 and 4, PvRK20-1 and three genes from the Arabidopsis genome project (Bevan et al., Unpublished data) here named as At1 (accession number CAB38617), At2 (accession number CAB81062) and At3 (accession number CAA18704). Cysteine amino acids are highlighted with black and the numbers above the double line indicate the number of amino acids between two cysteine residues. Putative glycosylation sites are highlighted with gray. Consensus indicates the conserved residues in all sequences shown.

FIG. 3 depicts Southern blot analysis of PRKs. Genomic DNA samples (5 μg) from S. tuberosum digested with the indicated restriction enzymes were separated by electrophoresis in a 0.8% agarose gel. Hybridization and washes were done according to Sambrook and Russell (2001). A fragment of the first 450 bases corresponding to the 5′ end of PRK-2 cDNA was used as probe labeled with [α-³²P]dCTP by random priming (Amersham International, UK). λ DNA digested with PstI together with a 100-basepair ruler were used as molecular markers.

FIG. 4 depicts the accumulation of PRK mRNAs in potato tissues in response to E. carotovora culture filtrate. (a) Accumulation of PRK mRNAs in leaves of Solanum tuberosum subsp. tuberosum cv. Bintje after treatment with culture filtrate (CF) from Erwinia carotovora subsp. carotovora strain SCC3193 (Pirhonen et al., 1988). Local treatment of potato leaves was done by applying 20-30 μl of CF to each leaf distributed in 4 to 6 different spots by gently pressing the tip of an automatic pipette against the leaf surface. (b) Accumulation of PRK transcripts in mini-tubers inoculated with 30-45 μl of CF applied by an automatic pipette. In (a) and (b), the amount of the corresponding RNA samples is indicated by a photo of the ethidium bromide-stained formaldehyde gels used for blotting. Potato plants used were grown axenically on MS medium (Murashige and Skoog 1962) for 3-4 weeks at 22° C. with a 14 hours light regime (100 to 150 μmol s⁻¹ m⁻²). In vitro plants grown for 3-4 weeks were either treated as indicated or transferred to soil and grown under gradually decreasing humidity but otherwise under similar conditions as indicated above for another ten days before treatment. Mini-tubers (1-3 grams fresh weight) were obtained from the soil plants grown under the same conditions for another 30-45 days. Three or more plants or mini-tubers were harvested after treatment at the indicated time points and total RNA was isolated (Verwoerd et al., 1989) and analyzed by RNA-gel blot experiments (Vidal et al., 1998). Each experiment was repeated twice or more. 10 μg of total RNA was used for each time point and hybridized with a 1 Kb PRK-4 probe corresponding to the extracellular domain, and labeled with [α-³²P]dCTP by random priming (Amersham International, UK).

FIG. 5 depicts the analysis of PRK mRNA accumulation in potato leaves after treatment with CF from E. carotovora subsp. carotovora and short oligogalacturonides. (a) Quantification of the PRK hybridization signals shown in b. The values shown are relative to the highest expression taken as 100%. Calculations were done using data obtained from Phosphor Imager (Fujifilm Bas-1500). (b) Accumulation of PRK mRNAs in potato leaves treated with 20-30 μl of the following: CF, 1 mM di-galacturonic acid (dimers) in water, 1 mM tri-galacturonic acid (trimers) in water, and H₂O which was used as a wound control. Dimers and trimers were purchased from Sigma (St. Louis, Mo.). The experimental conditions were otherwise as described in the legend to FIG. 4. (c) RT-PCR analysis of PRK-1 and 4. Leaf samples were treated as described in (b) and tuber samples as described in FIG. 4b. RT-PCR was performed as described by Sambrook and Russell (2001). For all samples, 1 μg of total RNA (DNA-free) was reverse transcribed in a final volume of 50 μl. The resulting cDNA was amplified by PCR using 1 μl of the RT reaction in a final volume of 50 μl and the following cycling conditions: 94° C. for 4 min; (94° C. for 30 s, 62° C. for 60 s, 72° C. for 60 s) 3 cycles; (94° C. for 30 s, 60° C. for 60 s, 72° C. for 60 s) 35 cycles; elongation step at 72° C. for 5 min. The primers used were 5′-CCAACCATGGCAGCTGTTGTTCTC-3′ for PRK-1 and PRK-4; 5′-CACGTACACTAAAAGTGGTACCAACAC-3′ for PRK-1 and 5′- AAGAGGGGTACGGAAGGAGTTC-3′ for PRK-4. The RT reaction with all the components but reverse transcriptase or without RNA were used as controls and did not give any bands after PCR amplification (data not shown).

FIG. 6 depicts the deduced amino acid sequence of PRK-1 with 676 amino acids (SEQ ID NO:1).

FIG. 7 depicts the deduced amino acid sequence of PRK-2 with 676 amino acids (SEQ ID NO:2).

FIG. 8 depicts the deduced amino acid sequence of PRK-3 with 651 amino acids (SEQ ID NO:3).

FIG. 9 depicts the deduced amino acid sequence of PRK-4 with 676 amino acids (SEQ ID NO:4).

FIG. 10 depicts the nucleic acid sequence of PRK-1 cDNA with 2201 nucleotides, accession number AJ306626 (SEQ ID NO:5). The poly(A)-tail is not included in the 2201 nucleotides.

FIG. 11 depicts the nucleic acid sequence of PRK-2 cDNA with 2225 nucleotides, accession number AJ306627 (SEQ ID NO: 6). The poly(A)-tail is not included in the 2225 nucleotides.

FIG. 12 depicts the nucleic acid sequence of PRK-3 cDNA with 2115 nucleotides, accession number AJ306628 (SEQ ID NO: 7). The poly(A)-tail is not included in the 2115 nucleotides.

FIG. 13 the nucleic acid sequence of PRK-4 cDNA with 2387 nucleotides, accession number AJ306629 (SEQ ID NO: 8). The poly(A)-tail is not included in the 2387 nucleotides.

FIG. 14 depicts the use Arabidopsis thaliana PRK. One full-length cDNA from the corresponding Arabidopsis gene has been isolated. The expression pattern of the Arabidopsis gene is similar to that of PRK responding to Erwinia carotovora.

FIG. 15 depicts the analysis of AtPRKs transgenic Arabidopsis plants. Transgenic Arabidopsis plants overexpressing this gene (sense) as well as transgenic plants where the expression of this gene is silenced (antisense) have been produced.

FIG. 16 depicts the sequence of Atpr3mia (SEQ ID NO: 9), sequenced by the present inventors and differing from the corresponding sequence in the databank with accession number CAA18465. It differs especially at the region of the transmembrane domain of the protein probably due to the computer programs used.

FIG. 17 depicts SEQ ID NO: 10 (top) and SEQ ID NO:11 (bottom).

FIG. 18 depicts SEQ ID NO: 12 (top) and SEQ ID NO: 13 (bottom).

FIG. 19 depicts table of similarities of the extracellular (C-terminal) domain.

FIG. 20 depicts amino acid sequence of Arabidopsis Atmia prk (SEQ ID NO: 14).

FIG. 21 depicts comparison between Atmia prk and databank sequence.

FIG. 22 depicts Atprks (Arabidopsis) ext and Stprks (Solanum tuberosum) extracellular cys similarity.

FIG. 23 depicts Atprks (Arabidopsis) ext and Stprks (Solanum tuberosum) extracellular cys similarity.

FIG. 24 depicts Atprks (Arabidopsis) ext and Stprks (Solanum tuberosum) extracellular cys similarity.

FIG. 25. Schematic illustration of the pPRKsense construct. The arrows indicate the orientation of the genes prk-2 and nptII, the latter encoding for neomycin phosphotransferase II, which confers kanamycin resistance. The expression of prk-2 is under control of 35S promoter from Cauliflower Mosaic Virus and the expression of nptII is under control of nopaline synthase (NOS) gene promoter. LB and RB represent the left and the right T-DNA border sequence, respectively.

FIG. 26. Schematic illustration of the pPRKantisense construct. The arrows indicate the orientations of the nptII gene, and the sense and antisense arms, which are constituted by an identical cDNA fragment sequence from prk-2. These arms are separated by an intron from the ppc1 gene from Solanum tuberosum, and the bracket indicate the DNA encoding an intron-spliced RNA with a self-complementary (hairpin) region, which is under control of 35S (2X) promoter from Cauliflower Mosaic Virus. The expression of nptII is under control of nopaline synthase (NOS) gene promoter. LB and RB represent the left and the right T-DNA border sequence, respectively.

FIG. 27. A fragment of prk-2 cDNA extra cellular domain. Bases representing start of translation (ATG) are underlined, and bases used to build the PRKantisense construct (sense and antisense arms; 100-387) are highlighted with gray. Numbers indicate the base positions from the prk-2 cDNA sequence.

FIG. 28. Intron sequence (877 bases) of phosphoenolpyruvate carboxylase (ppc1) gene from Solanum tuberosum used in the pPRKantisense construct (SEQ ID NO:15).

FIG. 29. Alignment of the cDNAs encoding PRKs at the region of the PRKantisense construct. Bases representing start of translation (ATG) are in bold letters, identical bases at the region used for sense and antisense arms of the PRKantisense construct are highlighted with gray and mismatching bases are highlighted with black. Numbers on the sides indicate the base positions or each cDNA sequence.

### DETAILED DESCRIPTION OF THE INVENTION

Definitions

In the present invention the terms used have the meaning they generally have in the fields of molecular biology, recombinant DNA technology, botany and plant pathology. Some terms, however, are used with a somewhat deviating or broader meaning. Accordingly, in order to avoid uncertainty caused by terms with unclear meaning some of the terms used in the specification and in the claims are defined in more detail below.

The term “ligand” means any molecule, that binds tightly and specifically to a macromolecule, usually but not necessary a protein, forming a macromolecule-ligand complex, or to a receptor forming a receptor-ligand complex.

The term “elicitor” means molecules produced by the presence or action of pathogen or pest or mechanical damage that induce a response by the host.

The term “receptor” means any protein that binds a specific extracellular signaling molecule (ligand) and then initiates a cellular response. Receptors can be located within the cell or in the plasma membrane with their ligand-binding domain exposed to the external medium.

The term “pathogen” means, but is not limited to bacteria, viruses, nematodes, fungi or insects (see e.g. Agrios, Plant Pathology, Academic Press, San Diego, Calif., 1997).

The term “PRK” means potato receptor-like kinase.

The term “Atprk” means potato receptor-like kinase from Arabidopsis.

The term “potato receptor like kinase (PRK)-like compounds” means compounds, which act as PRK-like proteins. They include polypeptides “substantially homologous” at amino acid level having a significant similarity or identity of at least 60%, more preferred embodiments include at least, 65%, 70%, 75%, 80%, most preferably more than 85% with the reference sequence.

The term “PRK-like compounds” means protein molecules or polypeptides being substantially homologous to PRK at amino acid level. Said “PRK-like molecules” are obtainable by isolation from natural sources. The PRK-like molecules are also producible by synthetic, semisynthetic, enzymatic and other biochemical or chemical methods including recombinant DNA techniques.

The term “PRK-like compounds” also comprises polypeptides having the structure, properties and functions characteristic of PRK-like proteins, including PRK-like proteins, wherein one or more amino acid residues are substituted by another amino acid residue. Also truncated, complexed or chemically substituted, forms of said PRK-like proteins are included in the term. Chemically substituted forms include for example, alkylated, esterified, etherified or amidized forms with a low substitution degree, especially using small molecules, such as methyl or ethyl, as substituents, as long as the substitution does not disturb the properties and functions of the PRK-like proteins. The truncated, complexed and/or substituted variants of said polypeptides are producible by synthetic or semisynthetic, including enzymatic and recombinant DNA techniques. The only other prerequisite is that the derivatives still are substantially homologous with and have the properties and/or express the functions characteristic of PRK-like proteins.

The term “PRK-like compounds” otherwise covers all possible splice variants of potato receptor like kinase (PRK). The PRK-like compounds can exist in different isoforms or allelic forms.

More specifically “PRK-like proteins” are substantially homologous with the amino acid sequence SEQ ID NO:1, SEQ ID NO:2, SEQ ID NO:3, SEQ ID NO:4 or SEQ ID NO:9.

The term “isoform” refers to the one of several forms of the same protein, whose amino acid sequences differ slightly but whose general activity is identical. “Isoforms” may originate from different sources, e.g. different plant species. Isoforms of PRK compounds can be generated by the cleavage. Different enzymatic and non-enzymatic reactions, including proteolytic and non-proteolytic reactions, are capable of creating truncated, derivatized, complexed forms of PRK proteins.

In the present invention the term “PRK-like compounds” includes nucleic acid sequences, which belong to the active PRK-like compounds of the present invention and which comprise isolated or purified “nucleic acid sequences” encoding PRK-like proteins or nucleic acid sequences with substantial similarity. They can be used as such or introduced into suitable transformation or expression vectors, which in turn can be introduced into suitable host organism to provide prokaryotic, eukaryotic organisms as well as transgenic plants capable of expressing altered levels of PRK-like proteins.

The term “nucleic acid sequences” refers to single of double-stranded polymer of deoxyribonucleotide or ribonucleotide bases. It includes chromosomal DNA, self-replicating plasmids, polymers of DNA or RNA. The “nucleic acid sequences” of the present invention are not in their natural state but are isolated and purified from their natural environment as transiently expressed mRNAs from a tissue. Thereafter the mRNAs are purified and multiplied in vitro in order to provide by technical means new copies, which are capable of encoding said PRK-like proteins. The nucleic acid sequences include both genomic sequences and cDNA.

The term “genomic sequence” means the corresponding sequence present in the nucleus of the plant cells and comprising introns as well as exons. In the present context the term “cDNA” means a DNA sequence obtainable by reversed translation of mRNA translated from the genomic DNA sequence including the complementary sequence.

The term “nucleic acid sequence encoding PRK or PRK-like proteins” means nucleic acid sequences encoding PRK or substantially homologous sequences. Said sequences or their complementary sequences or nucleic acid sequences containing said sequences or parts thereof, e.g. fragments truncated at the 3′-terminal or 5′-terminal end, as well as such sequences containing point mutations, are especially useful for as probes, primers and for preparing DNA constructs, plasmids and/or vectors useful for modulating the level of expression in plant tissues.

It is however clear for those skilled in the art that other nucleic acid sequences capable of encoding PRK-like proteins and useful for their production can be prepared. Said nucleic acid sequences and/or their complementary sequences should be capable of hybridizing under highly stringent condition (Sambrook and Russell, 2001) with SEQ ID NO:5, SEQ ID NO:6, SEQ ID NO:7 or SEQ ID NO:8.

The nucleic acid sequences of the present invention should have a substantial similarity with the sequences encoding PRK or PRK-like proteins. “Substantial similarity” in this context means that the nucleotide sequences fulfill the prerequisites defined above and have a significant similarity, i.e. a sequence identity of at least of at least 40%, more preferred embodiments include at least 45%, 50%, 55%, 60%, 65%, 70%, 75%, 80%, most preferably more than 85% with the reference sequence.

The term “nucleic acid sequences encoding PRK or PRK-like proteins” include their truncated or complexed forms as well as point mutations of said nucleic acid sequences as long as they are capable of encoding amino acid sequences having the essential structural features as well as the properties and/or functions of said PRK-like compounds.

The nucleic acid sequences are useful as such or inserted in transformation or expression vectors or host, said nucleic acid sequences being capable of encoding PRK or PRK-like proteins which are recognizable by binding substances specifically recognizing said PRK or PRK-like proteins. The nucleic acid sequences are useful in gene therapy or for preventing the genes causing the disease from expressing the gene products causing the diseases.

The PRK-like compounds include in addition to the proteins and nucleic acid sequences also binding substances.

The term “binding substances” means substances, which are capable of recognizing and specifically binding to natural PRK and/or PRK-like proteins or at least one specific portion of said molecules. Such binding substances are for example antibodies, receptors or ligands or proteins, specifically recognizing or binding to PRK or PRK-like proteins, ligands of PRK-like proteins or other binding proteins or peptides, comprising e.g. specific portions of said PRK-like compounds, but above all they mean antibodies capable of specifically recognizing one or more PRK-like compounds alone or in any combination. The antibodies include both polyclonal and/or monoclonal antibodies as well as fragments or derivatives thereof. Preferably, such binding substances, which recognize and bind to specific epitopes or active sites of the PRK-like compounds.

Said “binding substances” can be produced using specific domains of PRK-like compounds, their isomers as well as their fragments, derivatives and complexes with the prerequisite that they are capable of functioning in respective signaling pathway.

### GENERAL DESCRIPTION OF THE INVENTION

Identification of potato genes responsive to cell wall-degrading enzymes of Erwinia carotovora resulted in isolation of cDNA clones for four related receptor-like protein kinases. One of the putative serine-threonine protein kinases might have arisen through alternative splicing. These potato receptor-like kinases (PRK1-4) were highly similar (91-99%) most likely constituting a family of related receptors. All PRKs and four other plant receptor like kinases (RLKs) share in their extracellular domain a conserved bi-modular pattern of cysteine repeats distinct from that in previously characterized plant RLKs, suggesting they represent a new class of receptors. The corresponding genes were rapidly induced by E. carotovora culture filtrate (CF) both in the leaves and tubers of potato. Furthermore, the genes were transiently induced by short oligogalacturonides. The structural identity of PRKs and their induction pattern suggested that they constitute part of the early response of potato to E. carotovora infection.

The present inventors were interested in understanding potato (Solanum tuberosum) defense responses against E. carotovora. In order to isolate potato genes, which are involved in defense during the early stages of the plant-pathogen interaction, plants were inoculated with E. carotovora subsp. carotovora strain SCC3193 (Pirhonen et al., 1988) and pathogen induced cDNA clones were isolated by suppression subtractive hybridization (SSH) (see Birch et al., 1999 for methods). One of the 25 characterized cDNAs corresponding to CF-induced genes predicted a polypeptide showing similarity to protein kinases and was analyzed further. First the full-length cDNA corresponding to the original 206-bp cDNA fragment was isolated. To achieve this a cDNA library was constructed with RNA samples from CF-treated leaves using the SMART T™ RACE cDNA Amplification kit (Clonetech Laboratories, Inc). Screening of this library resulted in isolation of four cDNAs with different EcoRI-restriction patterns (data not shown) all homologous to the 206-bp cDNA fragment.

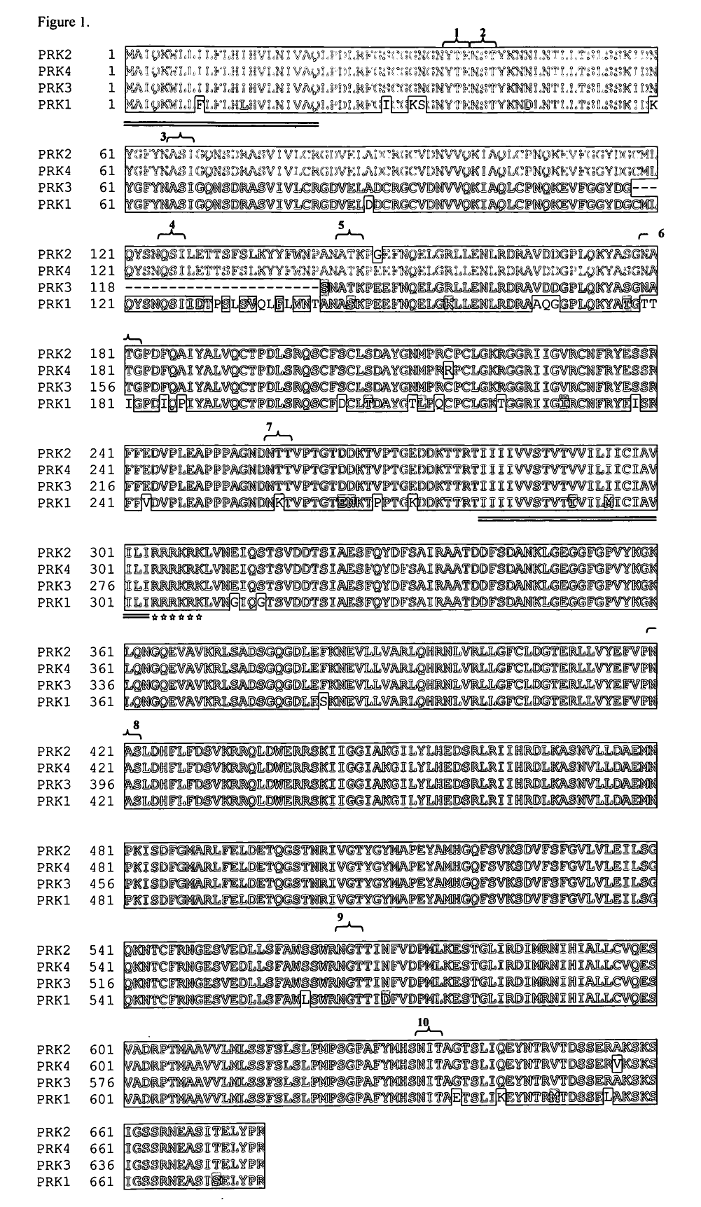

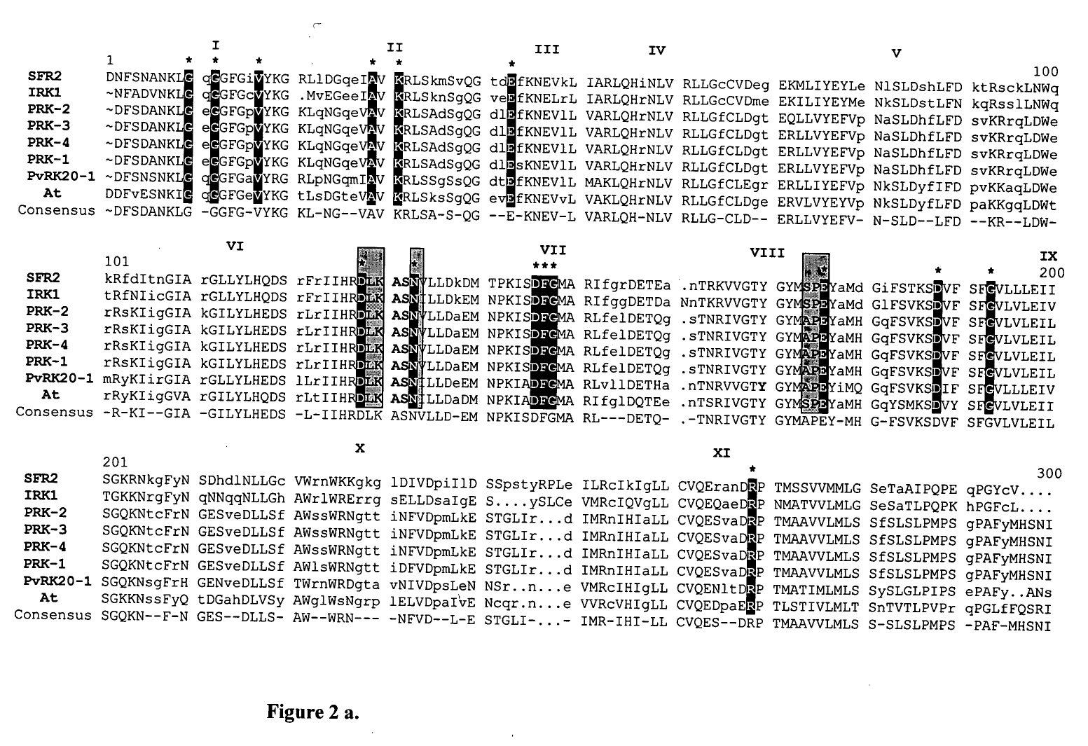

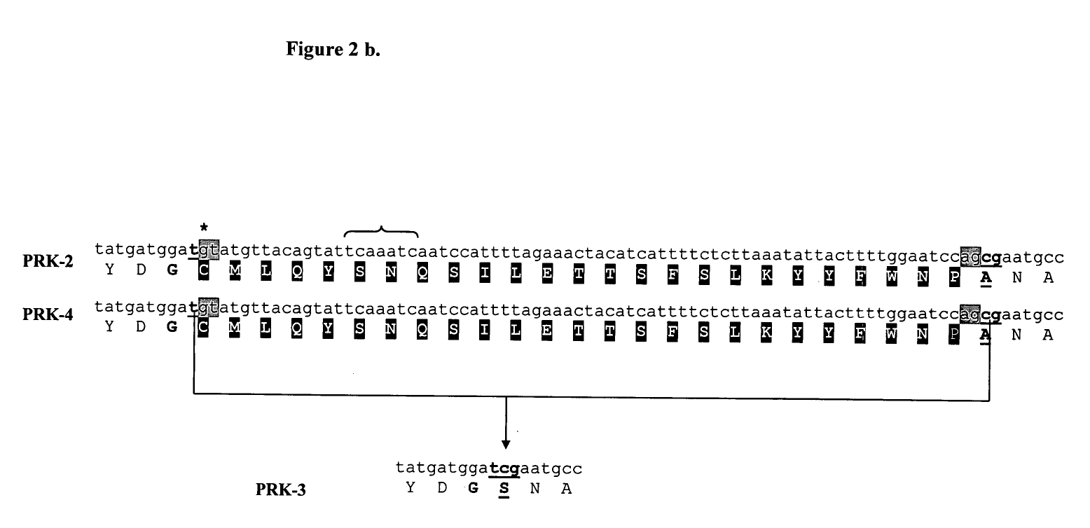

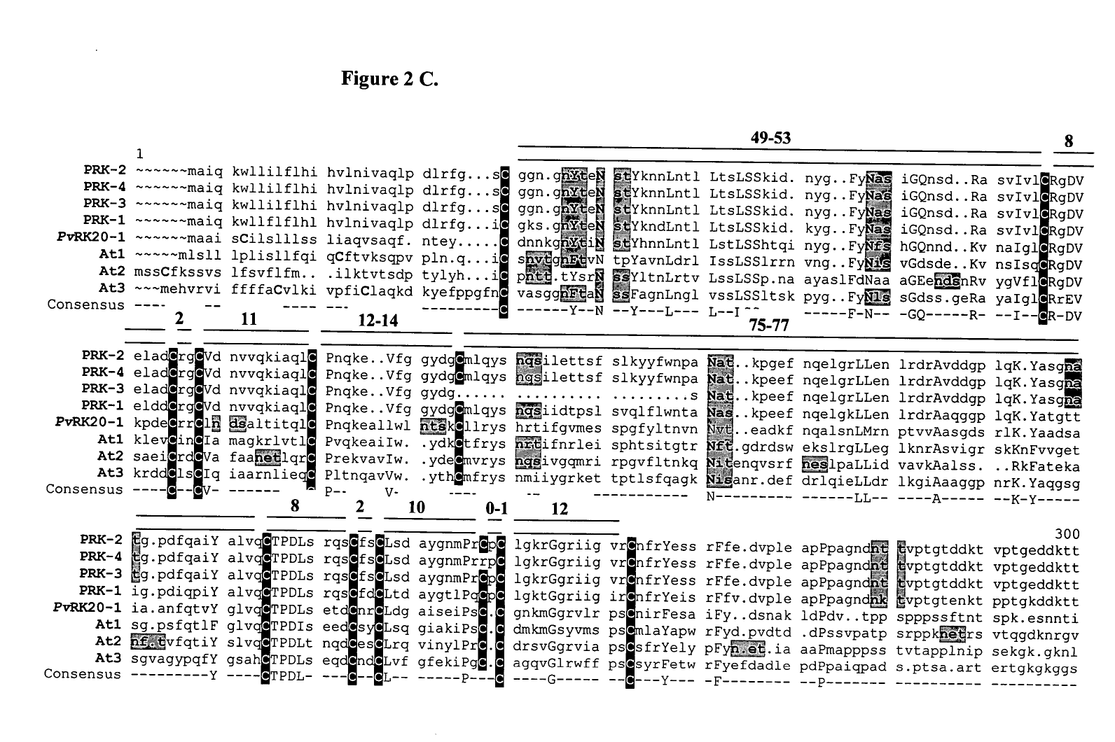

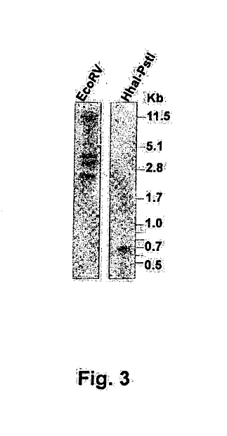

The four full-length cDNAs were designated PRK-1 (2201 nucleotides, accession number AJ306626), PRK-2 (2225 nucleotides, accession number AJ306627), PRK-3 (2115 nucleotides, accession number AJ306628), and PRK-4 (2387 nucleotides, accession number AJ306629) for potato receptor-like protein kinase (PRK). Their predicted open reading frames encoded 676 amino acid polypeptides with a calculated molecular mass of 75 kDa for PRK-1, 2 and 4 and a 651 amino acid polypeptide with a calculated molecular mass of 72 kDa for PRK-3 (FIG. 1). A hydropathy plot analysis (Kyte and Doolittle, 1982) indicated that the PRKs have two very hydrophobic regions (FIG. 1); one at the amino terminus indicative of a signal peptide (von Heijne, 1990), followed by a 255-280 amino acids hydrophilic domain that contains 6-7 putative glycosylation sites, and a second hydrophobic region of 23 amino acids which is followed by basic residues indicative of Type I integral membrane proteins (Singer, 1990). Alignment of their deduced amino acid sequences and comparison with similar sequences from databases showed that they were related of their C-terminal domains to plant receptor-like protein kinases (RLK), (FIG. 2A). The C-terminal domains of the four PRKs contain all the 11 subdomains conserved among different kinases (Hanks et al., 1988) including the 15 invariant amino acids with the right organization (FIG. 2A). In addition, the motifs in the catalytic core, DLKXXN in subdomain VI and APE in sub domain VIII are indicative of serine-threonine protein kinases (Hanks and Quinn, 1991). Characterization of the potato genome by EcoRV digestion, which does not cut the PRK cDNAs followed by Southern hybridization to a PRK specific probe (FIG. 3) suggested that there are probably at least 3 genes in this family. Although it remains to be biochemically confirmed, these data strongly suggest that the four potato PRKs form a family of receptor-like serine-threonine protein kinases.

PRK-2, 3 and 4 exhibit 98-99% amino acid similarity while PRK-1 shows 91-93% similarity to PRK-2, 3 and 4 (FIG. 1). The difference between PRK-1 and the other PRKs is accentuated when comparing the extracellular domains. The similarity of PRK-1 to PRK-2, 3 and 4 diminishes to 86-87% while the similarity between PRK-2, 3 and 4 is still 98-99%. Interestingly, PRK-3 presents a gap of 25 amino acids in a region of the putative extracellular domain that contains a conserved cysteine and a putative glycosylation site in the other PRKs (FIG. 1). Analysis of the corresponding cDNA sequences at this region revealed that the 25 amino acids difference of PRK-3 could be generated by alternative splicing from a different isoform (FIG. 2B). Identification of highly conserved sequences for splice sites (Brown and Simpson, 1998) flanking the 25 amino acids region strongly suggests the possibility for alternative splicing (FIG. 2B). This would result in removal of the codons for the 25 amino acids and as an additional consequence of such splicing to an amino acid substitution (serine instead of alanine) in PRK-3. Results of Southern analysis of the potato genome hybridized with a PRK probe specific to the fragment covering the putative intron supported this notion (FIG. 3). We could only detect a 700 bp HhaI and PstI fragment hybridizing to the probe but not a 625 bp fragment, which would be the expected size if the genomic DNA corresponding to PRK-3 would contain a deletion instead of an intron. Recently, it has been shown in Ipomea nil that alternative splicing occurred in a leucine-rich repeat receptor-like kinase (Bassett et al., 2000). Interestingly, a similar kind of splicing event was suggested in the extracellular domain of TGF-β type II receptors from mouse and human, which showed a 25 amino acids insertion containing one or two cysteines residues plus one putative glycosylation site, and one amino acid substitution at the splice junction (Hirai and Fujita, 1996; Suzuki et al., 1994). This splicing event resembles the one that can be predicted for the potato PRKs, suggesting that a similar mechanism could be involved in the processing of receptor-like serine-threonine protein kinases in different types of eukaryotic cells. This probably reflects the ability of cells to create receptors with the same function but different affinities for a ligand or structurally similar ligands.

Based on the structure of the putative extracellular domains, plant RLKs have been classified into several major classes (Braun and Walker, 1996; Walker, 1994). These include (i) the S-domain RLKs which contain extracellular domains homologous to the S-locus glycoproteins of Brassicaceae (Nasrallah et al., 1993; Walker and Zhang, 1990, Stein et al., 1991), (ii) the leucine rich repeat (LRR) RLKs such as Xa21 from rice, TMK1 and RLK5 from Arabidopsis (Chang et al., 1992; Song et al., 1995; Walker, 1993) and (iii) RLKs with the epidermal growth factor-like repeat (EGF) such as pro25 and the WAKs from Arabidopsis (He et al., 1999; Kohorn et al., 1992). Moreover, several RLKs with different types of extracellular domains have been identified recently (reviewed by Satterlee and Sussman, 1998). Comparison of PRKs with already known plant or other eukaryotic receptors, showed relation to PvPR20-1, a RLK of a new type from Phaseoulus vulgaris (Lange et al., 1999) and three different putative Arabidopsis RLKs (FIG. 2C). Alignment of their extracellular domains showed that: (i) the domains were 45 to 59% similar, i.e. 41 to 55% different, (ii) all of them contained 6-9 glycosylation sites except for one gene from Arabidopsis that had 4 glycosylation sites, (iii) the relative positions of 4 of the glycosylation sites were conserved and, (iv) all contained a conserved pattern of cysteine residues. This cysteine pattern presents two modules containing 6 cysteines each. The first module starts close to the putative signal peptide at the amino terminus and contains a C—X₍₄₉₋₅₃₎—C—X₍₈₎—C—X₍₂₎—C—X₍₁₁₎—C—X₍₁₂₋₁₄₎—C motif. It is followed by 75-77 amino acids that link it to the second module that contains a C—X₍₈₎—C—X₍₂₎—C—X₍₁₀₎—C—X₍₀₋₁₎—C—X₍₁₂₎—C motif, followed by a 42-46 amino acids segment before the putative transmembrane domain. Interestingly, PRK-3 lacks a cysteine in the first module while PRK-4 lacks a cysteine in the second module (FIG. 2C). Several eukaryotic receptors exhibit conserved cysteines, which could be involved in disulfide bond formation that may determine the general fold of the proteins. Furthermore, a similar cysteine knot structure has been described in different families of animal receptor kinases (McDonald and Hendrickson, 1993; Sun and Davies, 1995). On the other hand, different plant RLKs contain cysteine patterns (Chen, 2001; He et al., 1999; Kohorn et al., 1992; Satterlee and Sussman, 1998; Walker, 1994), but we failed to find the cysteine pattern described here in the extracellular domain of those sequences. Recently, Chen (2001) described a superfamily including a number of Arabidopsis RLKs and other proteins with C-rich repeats. Interestingly, part of the bi-modular cysteine pattern in PRKs described above (—C—X₍₈₎—C—X₍₂₎—C—) is also found in this superfamily of proteins. In conclusion, the structural similarities described and especially the conserved bi-modular cysteine pattern shared by the PRKs, PvPR20-1 from Phaseoulus vulgaris (Lange et al., 1999) and three different putative Arabidopsis RLKs, suggest that they represent a new class of plant RLKs.

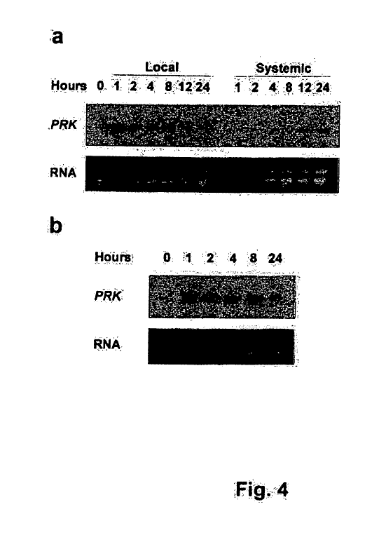

The expression of plant RLK genes have shown diverse patterns, while some of them displayed expression only in vegetative tissues (Kohorn et al., 1992), others were expressed only in reproductive tissues (Goring et al., 1992; Stein et al., 1991) and some have been shown in both vegetative and reproductive tissues (Pastuglia et al., 1997). Interestingly, some of the RLK genes are responsive to pathogens and elicitors, including PvRK20-1 from Phaseolus vulgaris (Lange et al., 1999), SFR2 from Brassica olearacea (Pastuglia et al., 1997), Wak1 (He et al., 1998) and RLKs (Du and Chen, 2000) from Arabidopsis thaliana and the disease resistance gene Xa21 from rice (Song et al., 1995). To elucidate the role of PRKs in plant response to E. carotovora we characterized the expression pattern of PRKs in different plant tissues after CF treatment of potato plants by RNA-gel blot hybridization (FIGS. 4a and b). The results show that leaf tissue treated locally with CF exhibits a fast accumulation of PRK transcripts with the highest level observed within one hour of treatment after which the level of mRNA decreased but stayed at elevated level up to 24 hours (FIG. 4a). The systemic leaves showed a very low and delayed induction of PRKs (FIG. 4a). A similar induction pattern to that of locally treated leaves was also observed in CF-treated potato mini-tubers (FIG. 4b). The early expression of PRK genes in response to CF-treatment strongly suggests that PRKs are involved in signal perception during potato defense responses against E. carotovora. Furthermore, the related structure and the related expression patterns of potato PRKs and PvRK20-1 suggest that these receptors could be involved in related cellular processes during the plant-pathogen interactions.

In order to demonstrate the function of the PRK family (PRK 1,2,3, and 4) in potato plants a set of potato plants were transformed to over express PRK-2, while another group of plants was engineered to silence the whole family of PRKs.

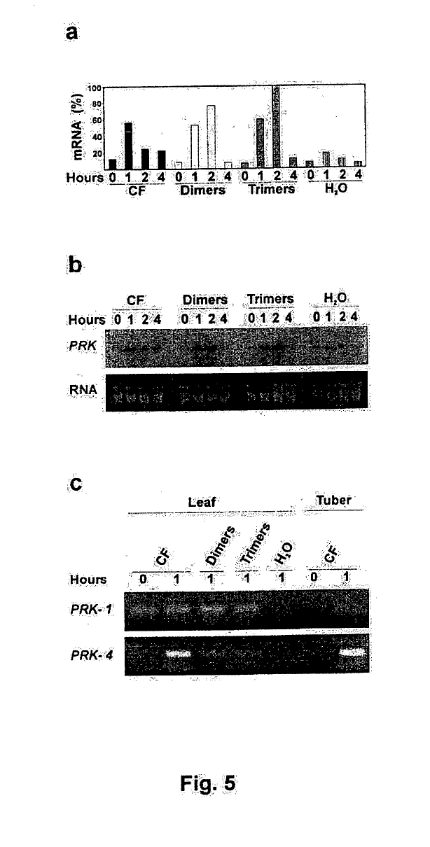

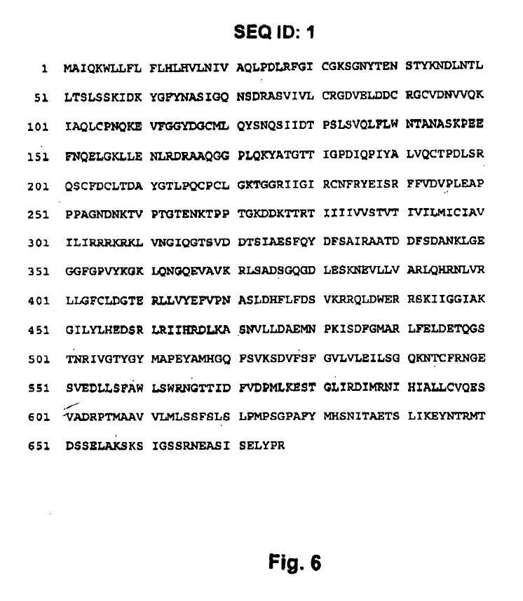

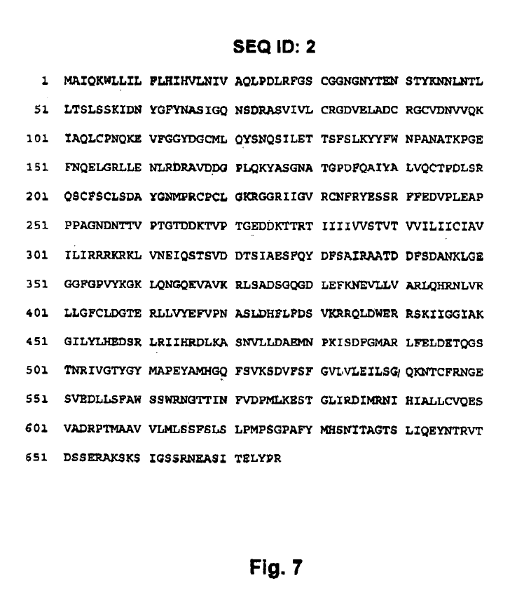

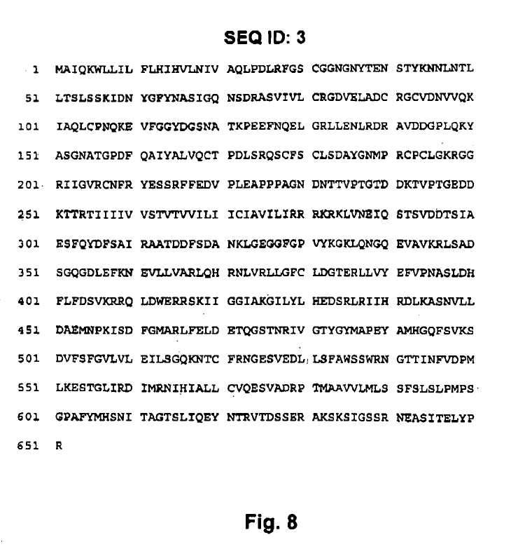

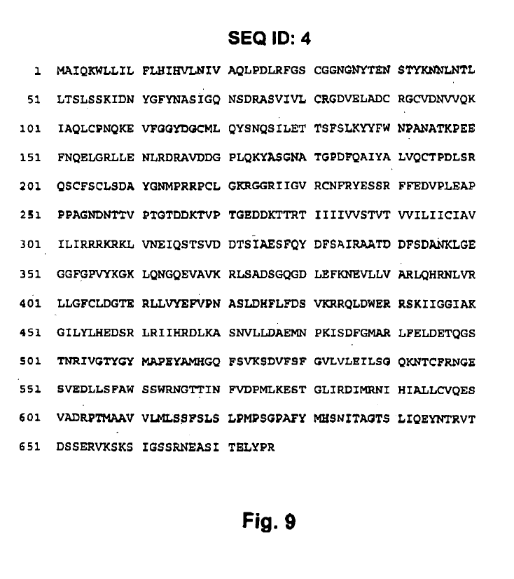

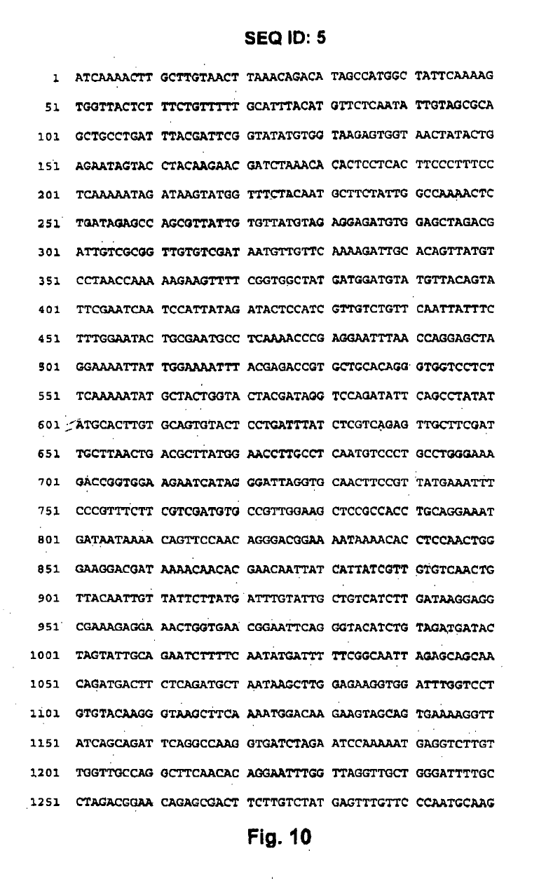

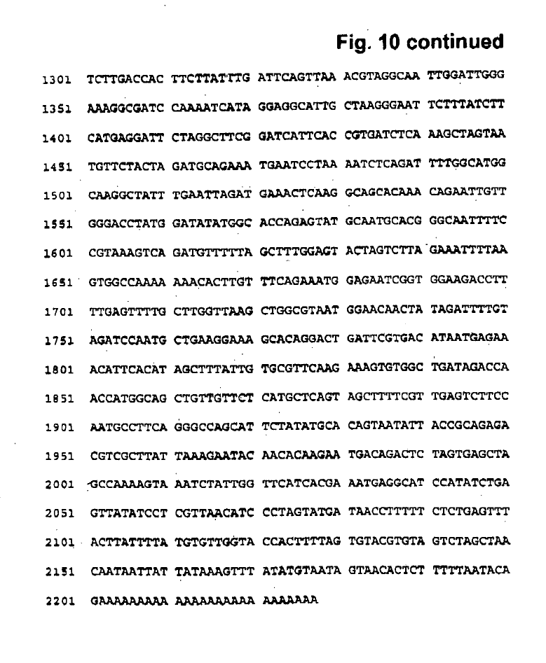

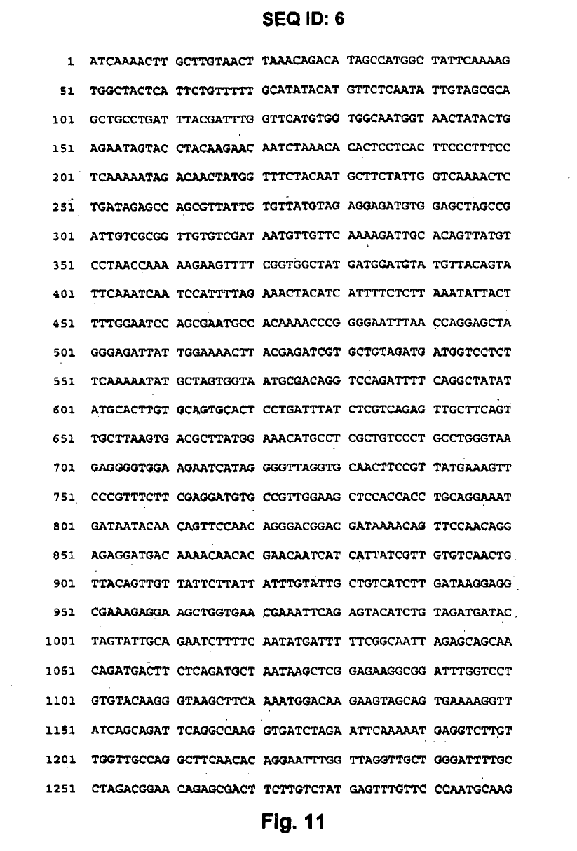

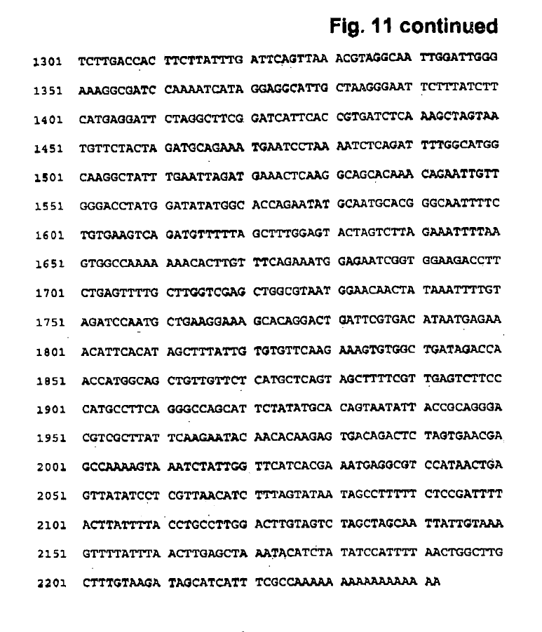

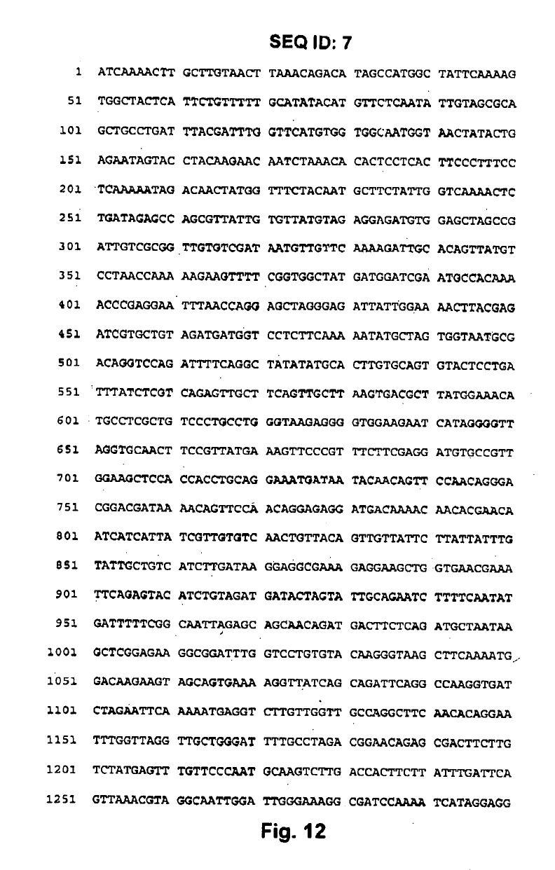

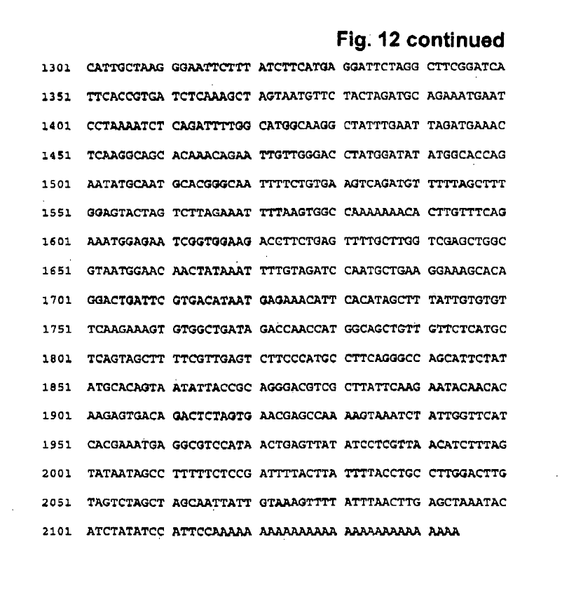

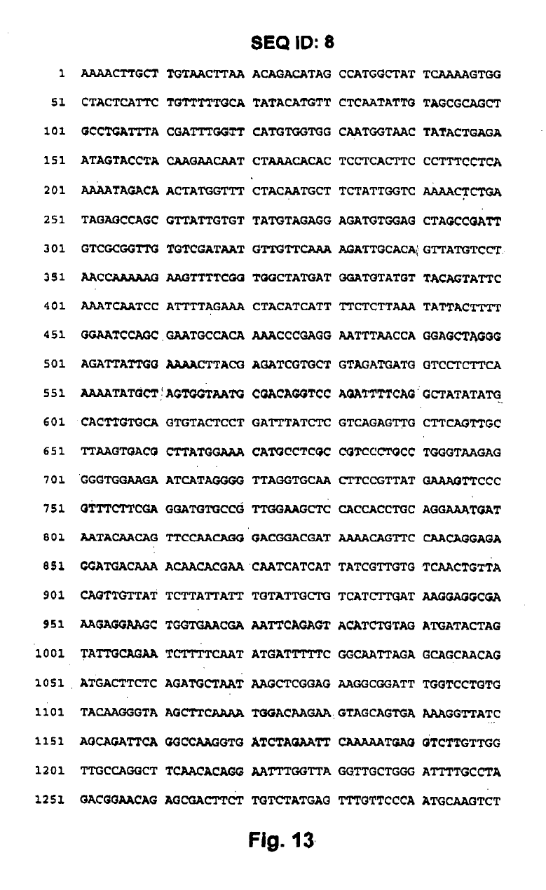

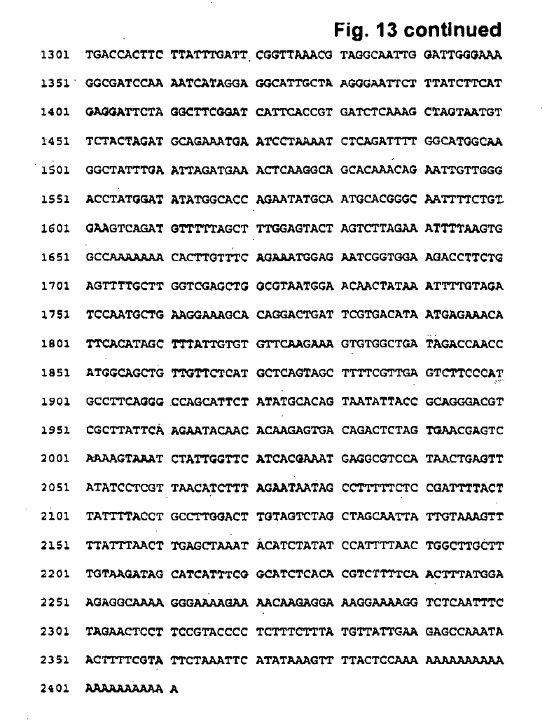

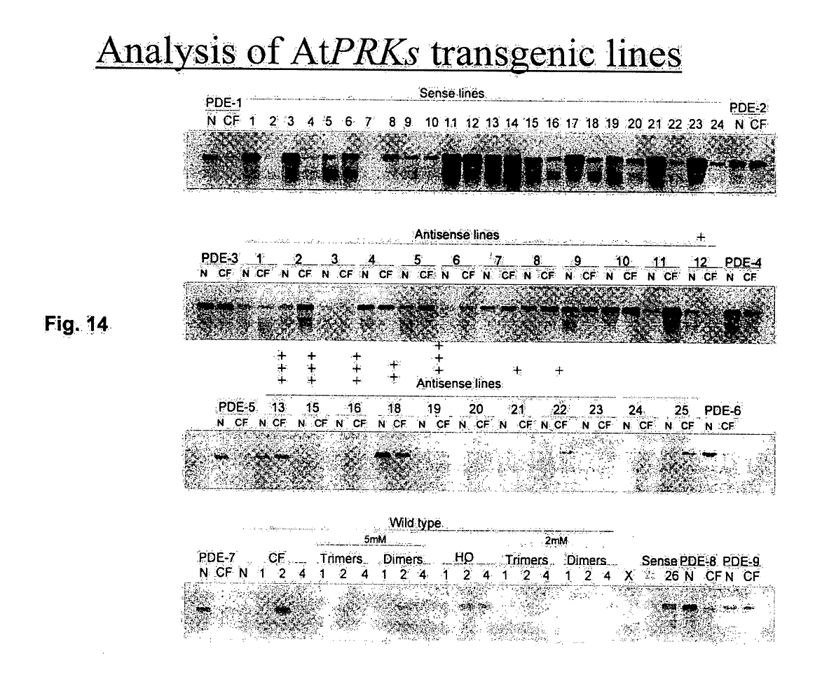

In plants, very little is known about the nature of the ligands interacting with serine-threonine RLKs. Recently, it has been shown that the extracellular domain of a RLK from Arabidopsis, BRI1, perceives brassinosteroids (He et al., 2000). On the other hand, in animals it has been shown that the interleukin 2 and the epidermal growth factor receptors are up regulated by their own ligands (Clark et al., 1985; Deeper et al., 1985). In order to elucidate the nature of the inducer of potato PRKs, we characterized accumulation of the corresponding transcripts following treatment with di-oligogalacturonic acid and tri-oligogalacturonic acid (FIGS. 5a and b), which have previously been shown to induce plant defense related genes responsive to E. carotovora (Norman et al., 1999). Plants treated with oligogalacturonides, showed a rapid but transient increase in PRK transcript levels while a very low induction was observed in water-treated wound control plants. This low but reproducible wound response (5a and b) could have been caused by a release from the plant of short oligogalacturonide elicitors during the treatment. The PRK transcripts were induced to similar levels (5 to 10-fold) during the first hour by both oligogalacturonides and CF. However; there was a distinct difference in expression patterns between CF and oligogalacturonide-treated samples at later time points. In the CF-treated samples, the level of PRKs was reduced during the second hour and continue unchanged at four hours, while plants treated with oligogalacturonides showed a higher level of induction during the second hour that was drastically decreased to control levels at four hours. The difference on the kinetics of PRK transcripts accumulation between CF and oligogalacturonide-treated plants might reflect the fact that the former contains an enzymatic solution, which is releasing different types of cell-wall fragments during several hours as maceration proceeds (data not shown) while the latter is a solution with a fixed concentration of oligogalacturonides. On the other hand, the drastic decrease of PRK mRNA levels at four hours of oligogalacturonide treatment may indicate the involvement of a different type of signal that controls the temporal regulation of PRK expression levels. RT-PCR was used to elucidate whether the different PRKs exhibited differences in their expression patterns (FIG. 5c). Due to extensive sequence similarities we could unambiguously distinguish only between PRK-1 and 4 but not between PRK-2 and 4. The results indicate that both genes PRK-1 and 4 are expressed similarly in response to CF and short oligogalacturonides in both leaf and tuber tissues, although we can not rule out the possibility of small differences in their expression patterns. PRK-3 specific PCR products were not detected suggesting a low level of expression.

In conclusion, the results of the expression studies demonstrate that short oligogalacturonides act as elicitors of PRK expression and indicate that E. carotovora released oligogalacturonides play an important role eliciting PRKs during the early stage of the potato-Erwinia interaction. Furthermore, the results suggest that the PRKs are involved in perception of E. carotovora by the host plant.

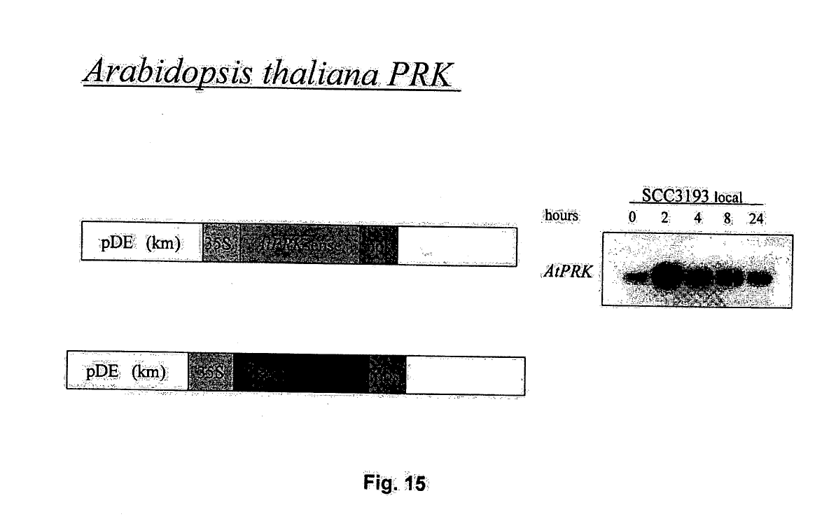

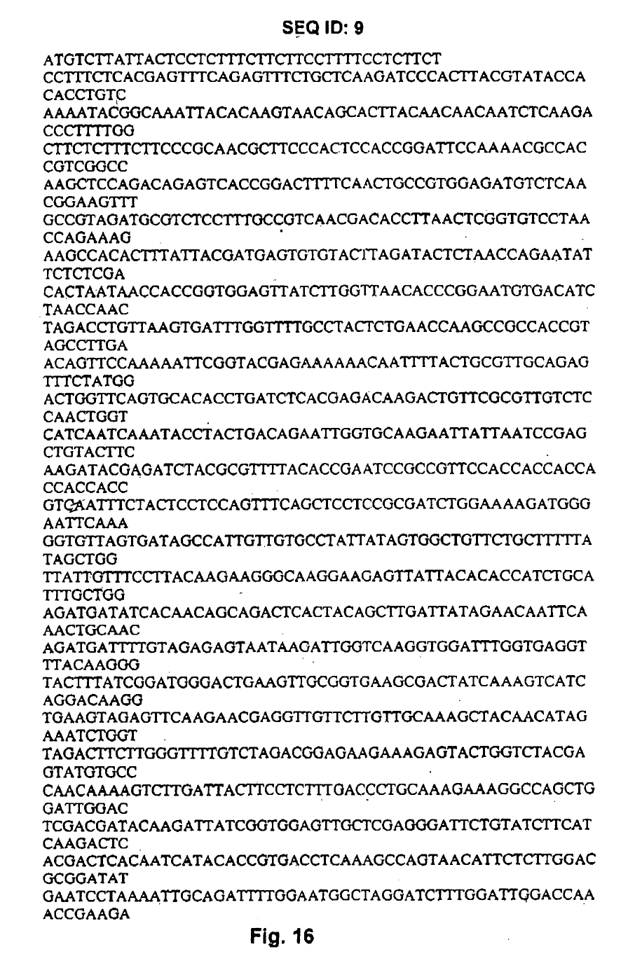

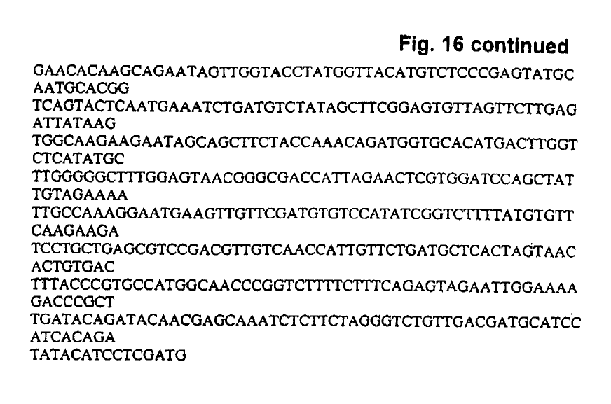

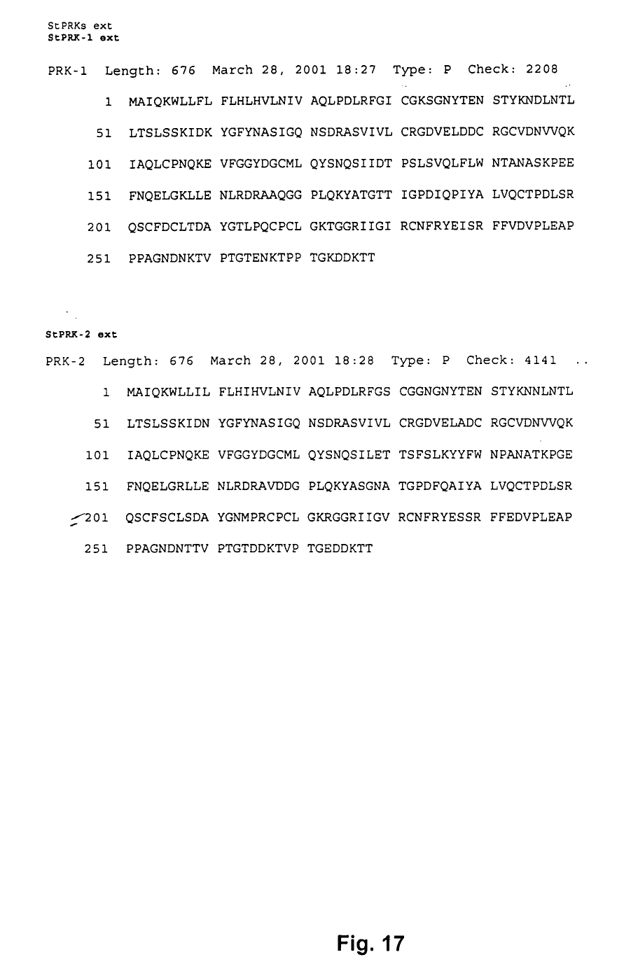

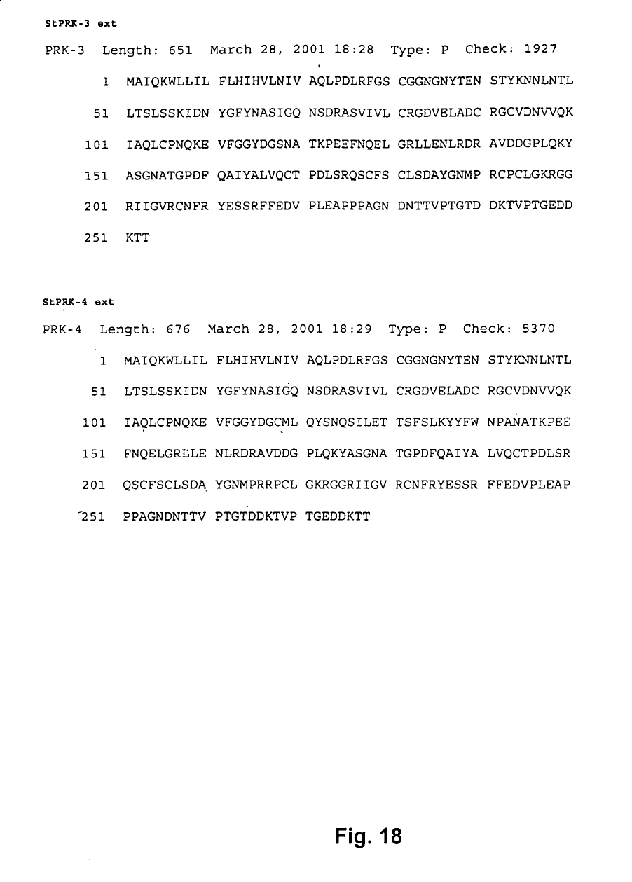

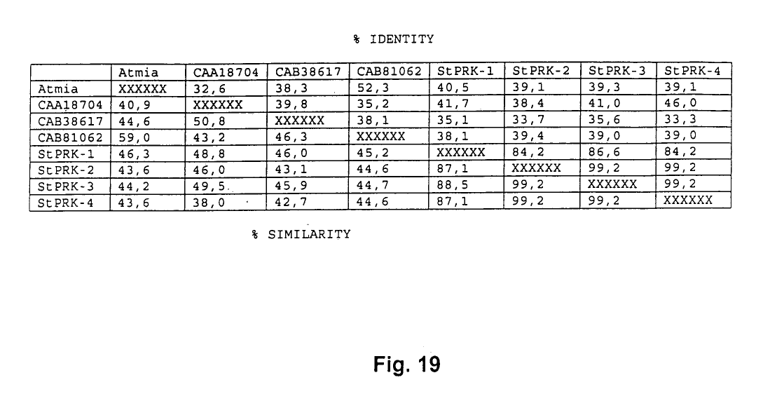

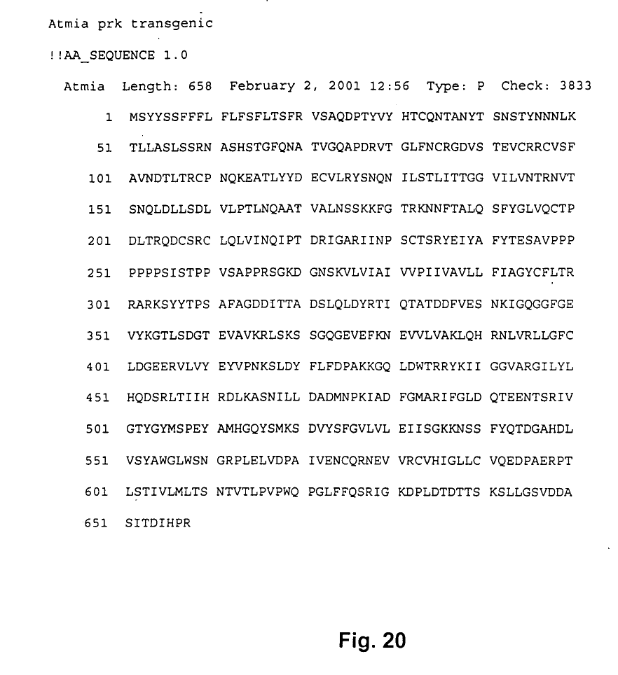

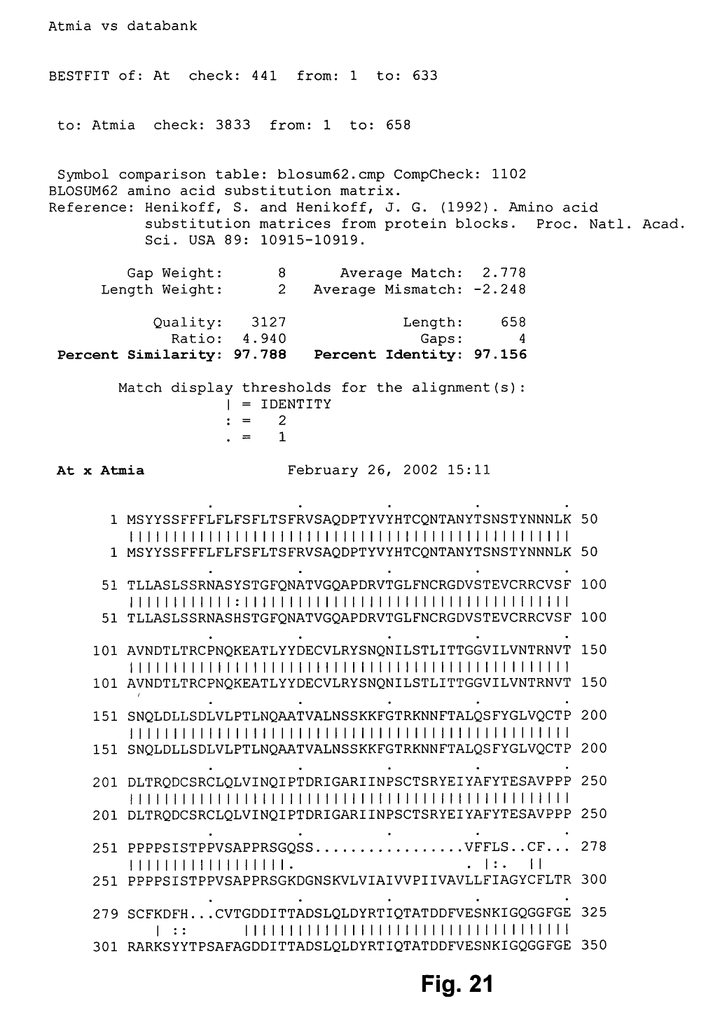

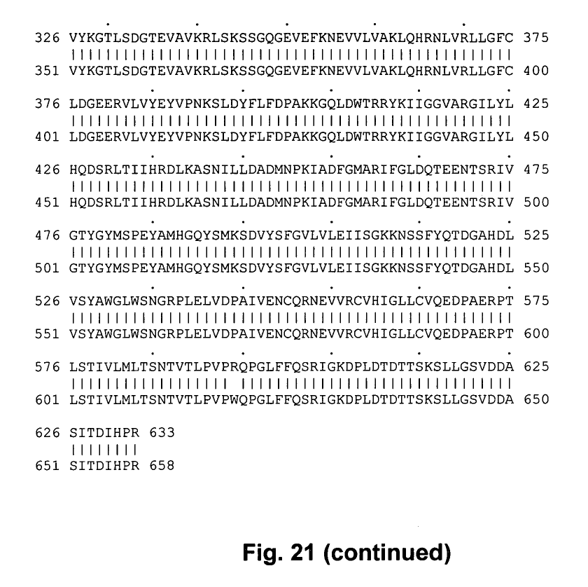

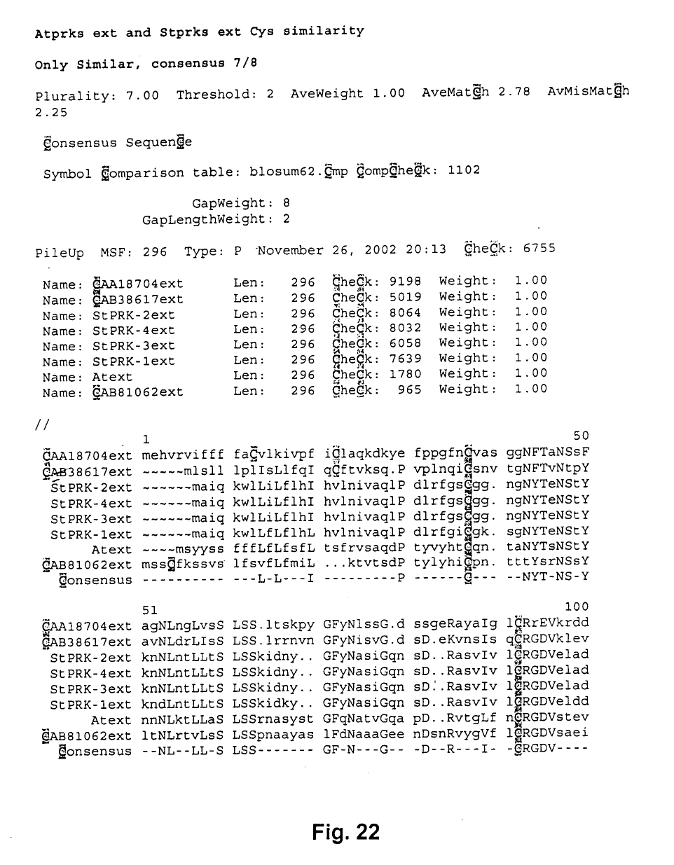

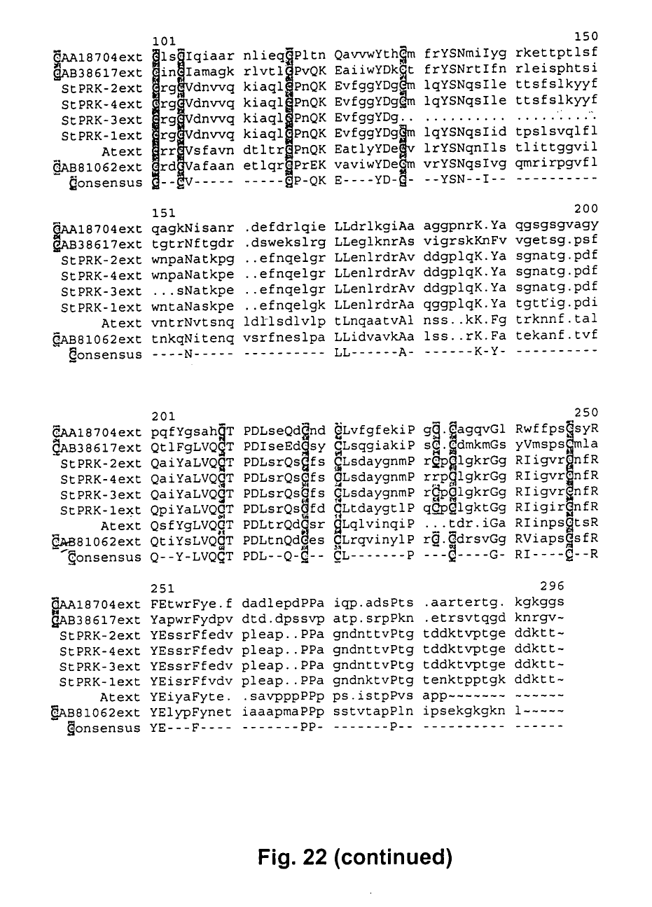

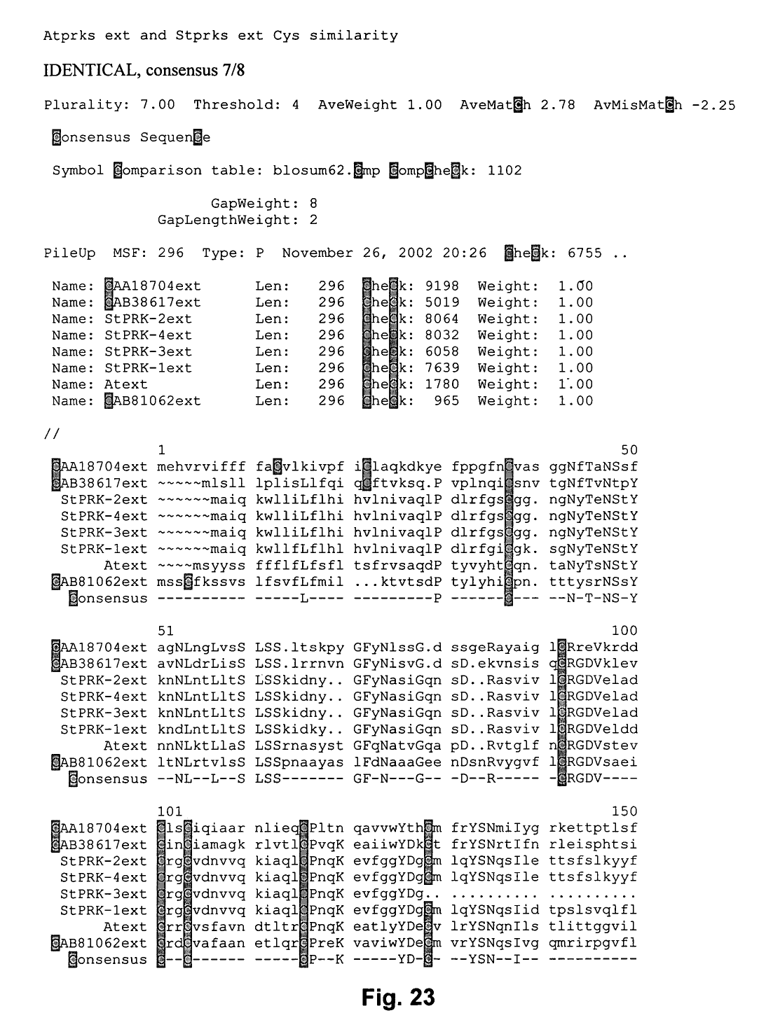

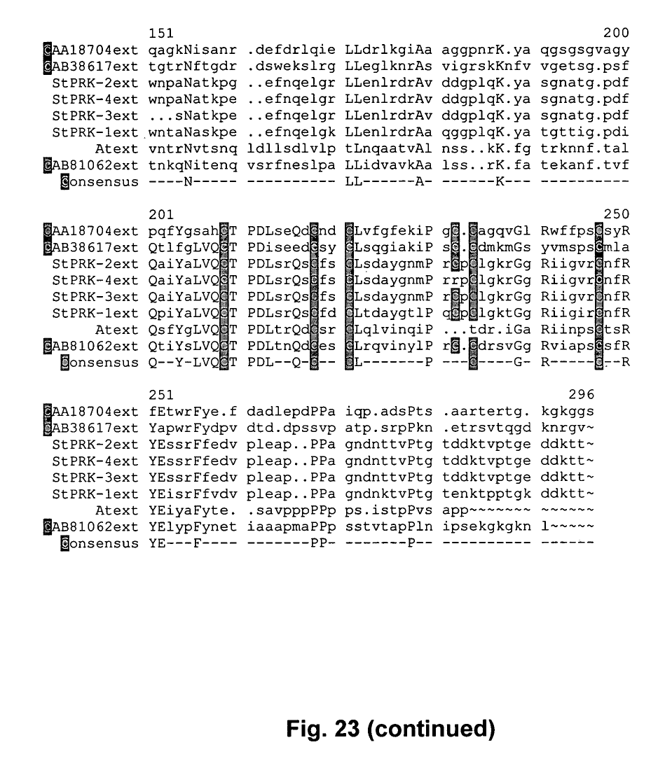

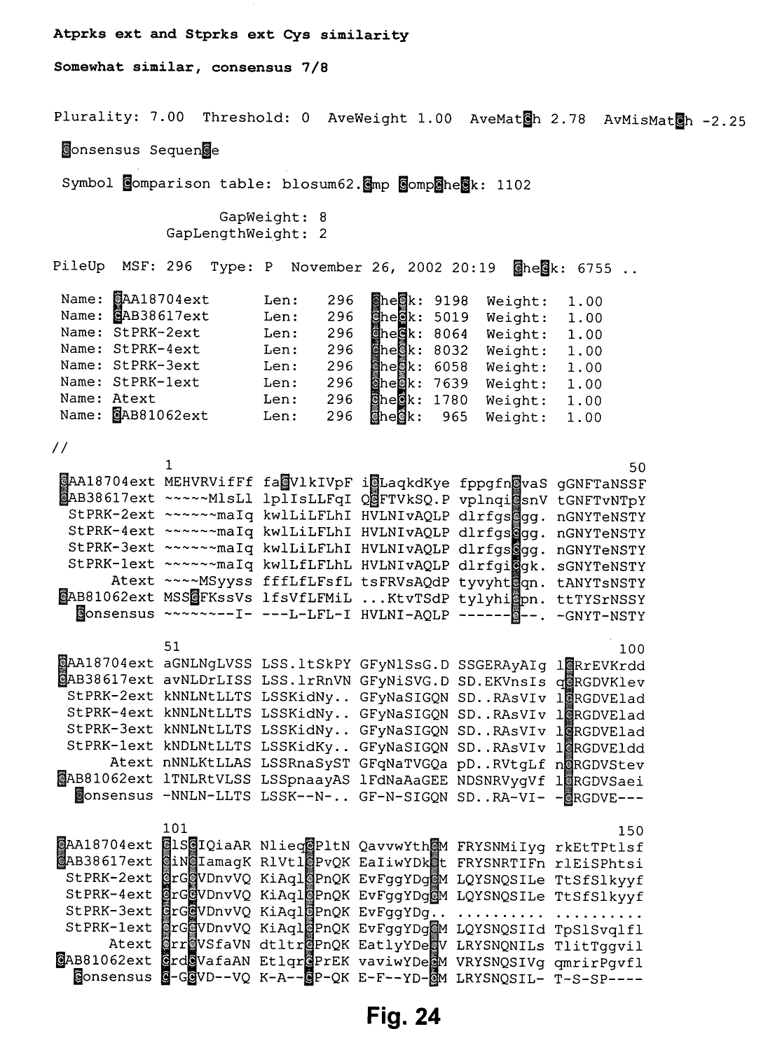

One full-length cDNA Atpr3mia (SEQ ID NO: 9) from the corresponding Arabidopsis gene was isolated. It differs from the corresponding sequence in the databank with accession number CAA18465. It differs especially at the region of the transmembrane domain of the protein probably due to the computer programs used. The expression pattern of the Arabidopsis gene is similar to that of PRK responding to Erwinia carotovora. Transgenic Arabidopsis plants overexpressing this gene (sense) as well as transgenic plants where the expression of this gene is silenced (antisense) (FIG. 15) have been produced.

The receptors of the present invention may also participate in other functions than conferring disease resistance for example regulation of growth in different plant species.

The following examples are intended for illustration of the present invention and should not be interpreted as limiting the present invention in any way.

### EXAMPLE 1

Atmia prk Transgenic Arabidopsis Infected with Erwinia Carotovora Subsp. Carotovora SCC1

Arabidopsis thaliana plants were transformed using the Agrobacterium-mediated transformation method of Clough and Bent (1999, Plant Journal 16, 735-743). The Arabidopsis plants were transformed using vacuum infiltration method without plant tissue culture or regeneration. Developing floral tissues were dipped into a solution containing Agrobacterium tumefaciens, 5% sucrose and 500 microliters per litre of surfactant Silwet L-77. Plant tissue culture media, the hormone benzylamino purine and pH adjustment were unnecessary.

Arabidopsis plants were transformed with Atmia prk gene (SEQ ID NO:9) and they were subsequently infected with Erwinia carotovora subsp. carotovora SCC1. 3-week old seedlings of Arabidopsis transgenic lines where the Arabidopsis homolog of PRK (Atmia prk) is overexpressed (S 2142, S 1713) or silenced with antisense constructs (AS 1961, AS 1564) were used. The number of plants exhibiting disease symptoms over those locally inoculated with different size of inocula of the Erwinia strain SCC 1 are presented in Table 1.

TABLE 1
Plants exhibiting disease symptoms (at 96 hrs)/total of treated plants

| | SCC1 inoculum. | | | | |
|---|---|---|---|---|---|
| | S 2142 | S 1713 | AS 1961 | AS 1564 | PDE control |
| 50.000 CFU | 1/16 | 1/12 | 9/24 | 7/17 | 3/17 |
| 20.000 CFU | 1/18 | nt | 5/18 | 9/18 | 5/18 |
| 7.500 CFU | 2/18 | 1/12 | 8/12 | 5/17 | 3/12 |

nt not tested

The PDE control indicates transgenic Arabidopsis harboring the transformation vector without any PRK insert. The plants were locally inoculated without wounding and the development of disease symptoms assessed 96 hrs post inoculation.

The results demonstrate that plants overexpressing the Atmia prk show enhanced disease resistance [only 4/52 (S 2142) and 2/24 (S 1713) plants with disease symptoms] when compared to the vector PDE control (11/47 plants with disease symptoms). In contrast, plants where the gene is silenced by antisense constructs are more sensitive to the pathogen (22/54 (AS 1961) and 21/52 (AS 1564) plants with disease symptoms.

In conclusion, the results indicate, that the Atmia prk gene is required for full disease resistance in Arabidopsis. The results also indicate that overexpression of the Atmia prk gene enhances disease resistance.

The overexpression of Atmia prk gene or genes can be used to enhance disease resistance in various agricultural plants, in particular disease resistance to necrotrophic pathogens and possibly herbivores. Another application could be additional enhancement of the resistance of such transgenic lines by adding chemical preparations (e.g. by spraying fields) containing oligouronides. The above mentioned preparations might also be used to enhance resistance of normal (not transformed with prk) plants.

### EXAMPLE 2

PRK-2 Over Expression in Potato Plants

In order to demonstrate the role of PRKs in potato plants and to assess their role in plant defense an economically important potato cultivar (Solanum tuberosum cv Bintje) was engineered to overexpress PRK-2.

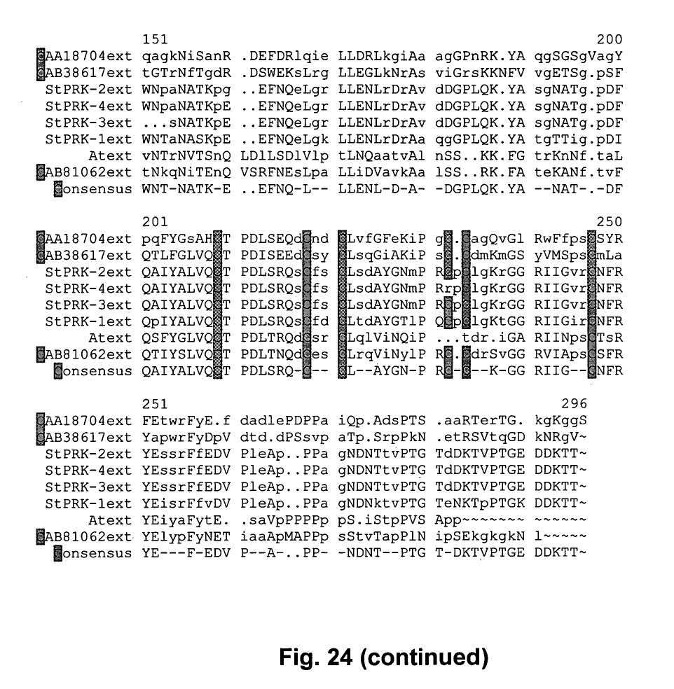

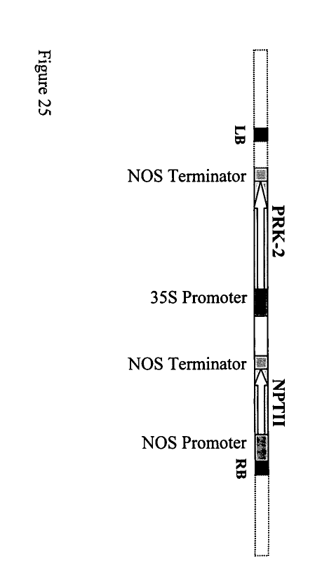

The full length cDNA of prk-2 was cloned in sense orientation into plasmid pROK2, which contains 35S promoter of CaMV in the binary vector pBIN19, resulting in pPRK2sense construct. The sense orientation of prk-2 under control of 35S CaMV promoter was confirmed by sequencing. A schematic illustration of the final construct is depicted in FIG. 25. All DNA manipulation and cloning was performed by established procedures described in Sambrook et al. (1989).

For genetic transformation, Agrobacterium tumefaciens strain C58C1 (rifampicin resistant) containing the disarmed nopaline Ti-plasmid pGV3850 that confers resistance to carbenicilin (Zambryski et al 1983) was transformed by standard procedures with pPRK2sense construct conferring additional resistance to kanamycin.

Agrobacterium-mediated genetic transformation of potato for over expression of PRK2 was performed essentially as described by Beaujean et al. (1998). Solanum tuberosum cv. Bintje plants were grown axenically on MS medium (Murashige and Skoog, 1962) at 22° C. under a 16 hours light period (100-150 μumol/s/m²).

Plants were excised with sharp scalpel and internodal explants (4-6 mm) were used for transformation with Agrobacterium tumefaciens containing pPRK2sense construct. Such internodal explants were wounded lengthwise with a scalpel, incubated for 30 minutes in a Petri dish with MS liquid medium containing 1:10 vol. of bacterial suspension, blotted dry on a filter paper and cultured on callus inducing medium (CIM) (Table 2 below) under the conditions described above. After three days of co-cultivation, the explants were washed for 30 minutes with MS liquid medium containing 1 g/l cefotaxime. Then, the explants were dry blotted and placed on selection medium (CIM containing 250 mg/l cefotaxime and 125 mg/l kanamycin).

After the callus is well developed the plants are subcultured on Shoot Induction Medium (SIM) (Table 2) and then transferred to jars containing selective rooting medium (RIM) (Table 2). Only plantlets with well developed roots are transferred to MS and subjected to primary molecular analysis including PCR and northern blot to confirm transformation.

Table 2. Composition of different culture media used during genetic engineering of potato.

CIM

MS salts 4.71 g/l

Sucrose 30 g/l

MES: 2-(N-morpholino) ethanesulphonic acid 0.5 g/l

Zeatine riboside (cytokinin) 0.8 mg/l

2,4-D (auxin) 2 mg/l

agar 8.5 g/l

pH a 5.7

SIM

MS salts 4.71 g/l

Sucrose 30 g/l

MES: 2-(N-morpholino) ethanesulphonic acid 0.5 g/l

Zeatine riboside (citokinin) 0.8 mg/l

GA₃ 3 mg/l

pH a 5.7

agar 8.5 g/l

Claforan 500 mg/l

Kanamycin 50 mg/l

RIM

MS salts 4.71 g/l

Sucrose 30 g/l

MES: 2-(N-morpholino) ethanesulphonic acid 0.5 g/l

IBA (auxin) 0.1 mg/l

pH a 5.7

agar 8.5 g/l

Claforan 500 mg/l

Kanamycin 50 mg/l

### EXAMPLE 3

Silencing PRK Expression in Potato Plants

Constructs encoding RNA with regions of self-complementary sequences (arms), which form the stem of a hairpin structure, efficiently induce gene silencing through such hairpin-stem sequences of the RNA molecule (Smith et al., 20000; Wesley et al, 2001). This type of constructs containing DNA sequences ranging from 98 to 800 bases cloned as inverted-repeat arms, have been shown to successfully induce gene silencing in several plant species (Wesley et al., 2001).

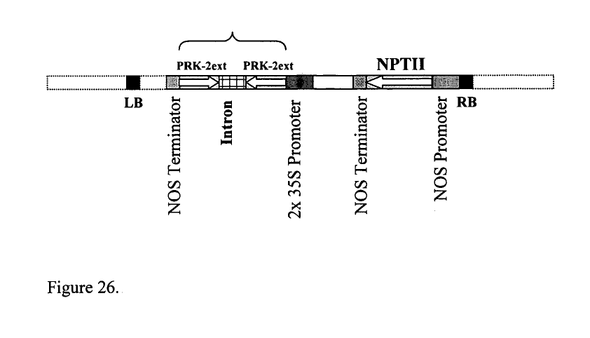

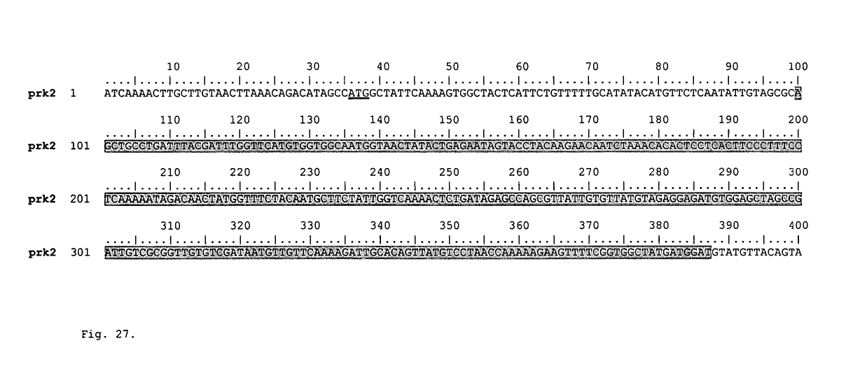

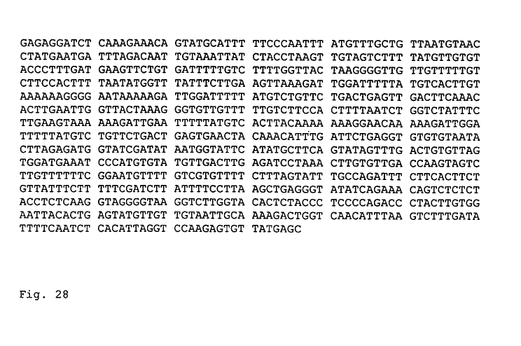

In order to specifically silence the whole PRK family a construct called pPRKantisense (FIG. 26) was constructed. The pPRKantisense encodes an intron-spliced RNA with a self-complementary (hairpin stem) region, under the control of the 35S promoter (2x) of CaMV. The construct contains a sense and an antisense arms (identical sequences but in inverted orientation) constituted by a cDNA segment from prk-2 (nucleotides 100 to 387, encoding a part of the extra cellular domain of the protein) (FIG. 27). The arms are separated by an intron sequence of the phosphoenolpyryvate carboxylase (ppc1) gene from Solanum tuberosum (AC number X90982) (FIG. 28).

Agrobacterium tumefaciens strain C58C 1 (rifampicin resistant) containing the disarmed nopaline Ti plasmid pGV3850 conferring carbenicillin resistance was transformed by standard procedures with pPRKantisense construct.

The potato plants (Solanum tuberosum cv Bintje) were transformed with the PRKantisense construct by Agrobacterium-mediated transformation similarly as described in Example 1.

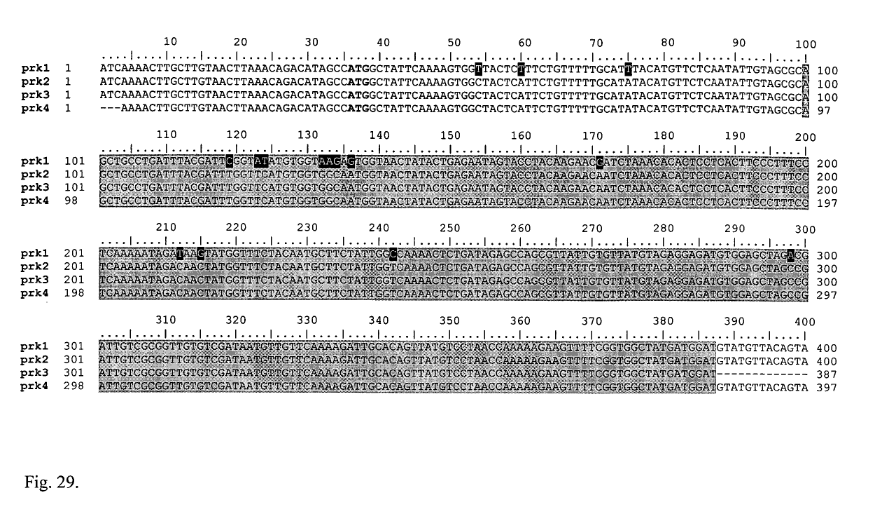

Because the DNA fragment from prk-2 used in this construct corresponds to the region encoding the extra cellular domain of PRK-2 and not the kinase domain, which could be eventually silenced by unrelated kinases and because the sequence homology of the DNA fragment from prk-2 used in this construct is 100% identical with prk-3 and prk-4, and 96% identical with prk-1 (FIG. 29) the PRKantisense construct is substantially silencing the whole family of PRKs form Solanum tuberosum.

### EXAMPLE 4

Characterization of the Transgenic Potato Plants

Potato plants transformed with either prk2sense construct of prkAntisense constructs are infected with Erwinia carotovora subs. Carotovora SCC1. Disease symptoms of the locally inoculated plants are recorded from the plants and compared with untransformed control plants or plants transformed with the transformation vector without a prk2 insert or pPRKantisense insert.

### LITERATURE CITED

Bassett, C. L., Nickerson, M. L., Cohen, R. A., and Rajeevan, M. S. (2000) Alternative transcript initiation and novel post-transcriptional processing of a leucine-rich repeat receptor-like protein kinase gene that responds to short-day photoperiodic floral induction in morning glory (Ipomea nil). Plant Mol. Biol. 43, 43-58.
Beaujean, A., Sangwan, R. S., Lecardonnel, A. and Sangwan-Norreel, B. S. (1998) Agrobacterium-mediated transformation of three economically important potato cultivars using sliced internodal explants: an efficient protocol of transformation. J. Exp. Botany 49: 1589-1595.
Birch, P. R. J., Avrova, A. O., Duncan, J. M., Lyon, G. D., and Toth R. L. (1999) Isolation of potato genes that are induced during an early stage of the hypersensitive response to Phytophthora infestans. Molec. Plant-Microbe Interact. 12, 356-361.
Braun, D. M. and Walker, J. C. (1996) Plant transmembrane receptors: new pieces in the signaling puzzle. Trends Biochem. Sci. 21, 70-73.
Brown, J. W. S. and Simpson, C. G. (1998) Splice site selection in plant pre-mRNA splicing. Annu. Rev. Plant Physiol. Plant Mol. Biol. 49, 77-95.
Chang, C., Schaller, G. E., Pattersson, S. E., Kwok, S. F., Meyerowitz, E. M., and Bleecker, A. B. (1992) The TMK1 gene from Arabidopsis codes for a protein with structural and biochemical characteristics of a receptor protein kinase. Plant Cell 4, 1263-1271.
Chen, Z. (2001) A superfamily of proteins with novel cysteine-rich repeats. Plant Physiol. 126, 473-476.
Clark, A. J. L., Ishii, S., Richert, N., Merlino, G. T., and Pastan, I. (1985) Epidermal growth factor regulates the expression of its own receptor. Proc. Natl. Acad. Sci. USA 82, 8374-8378.
Clough, S. J. and Bent, A. F. (1999) Floral dip: a simplified method for Agrobacterium-mediated transformation of Arabidopsis thaliana Plant Journal 16, 735-743.
Collmer, A. and Keen, N. T. (1986) The role of pectic enzymes in plant pathogenesis. Annu. Rev. Phytopathol. 24, 383-409.
Davis, K. R., Lyon, G., D., Darvill, A., G., and Albersheim, P. (1984) Host pathogen interactions. XXV. Endopolygalacturonic acid lyase from Erwinia carotovora elecits phytoalexin accumulation by releasing plant cell wall fragments. Plant Physiol. 74, 52-60.
Davis, K. R. and Ausubel, F. M. (1989) Characterization of elicitor-induced defense responses in suspension-cultured cell of Arabidopsis. Mol. Plant-Microbe Interact. 2, 363-368.
Deeper, J. M., Leonard, W. J., Drogula, C., Krönke, M., and Waldmann, T. A. (1985) Interleukin 2 (IL-2) augments transcription of the IL-2 receptor gene. Proc. Natl. Acad. Sci. USA 82, 4230-4234.
Du, L. and Chen, Z. (2000) Identification of genes encoding receptor-like protein kinases as possible targets of pathogen- and salicylic acid-induced WRKY DNA-binding proteins in Arabidopsis. Plant J. 26, 837-847.
Goring, D. R. and Rothstein, S. J. (1992) The S-locus receptor kinase gene in a self-incompatible Brassica napus line encodes a functional serine/threonine kinase. Plant Cell 4, 1273-1281.
Hanks, S. K., Quinn, A. M., and Hunter, T. (1988) The protein kinase family: conserved features and deduced phylogeny of the catalytic domain. Science 241, 42-52.
Hanks, S. K. and Quinn, A. M. (1991) Protein kinase catalytic domain sequence data base: identification of conserved features of primary structure and classification of family members. Methods Enzymol. 200, 38-62.
He, Z.-H., He, D., and Kohorn, B. D. (1998) Requirement for the induced expression of a cell wall associated receptor kinase for survival during the pathogen response. Plant J. 14, 55-63.
He, Z.-H., Cheeseman, I., He, D., and Kohorn, B. D. (1999) A cluster of five cell wall-associated receptor kinases, Wak1-5, are expressed in specific organs of Arabidopsis. Plant Mol. Biol. 39, 1189-1999.
He, Z., Wang, Z.-Y., Li, J., Zhu, Q., Lamb, C., Ronald, P., and Chory, J. (2000) Perception of brassinosteroids by the extracellular domain of the receptor kinase BRI1. Science 288, 2360-2363.
von Heijne, G. (1990) The signal peptide. J. Membr. Biol. 115, 195-201.
Hirai, R. and Fujita, T. (1996) A human transforming growth factor-β type II receptor that contains an insertion in the extracellular domain. Exp. Cell Res. 223, 135-141.
Kohorn, B. D., Lane, S., and Smith, T. A. (1992) An Arabidopsis serine/threonine kinase homologue with an epidermal growth factor repeat selected in yeast for its specificity for a thylakoid membrane protein. Proc. Natl. Acad. Sci. USA 89, 10989-10992.
Kotoujansky, A. (1987) Molecular genetics of pathogenesis by soft-rot Erwinias. Annu. Rev. Phytopathol. 25, 405-430.
Kowyama, Y., Kakeda, K., Kondo, K., Imada, T., and Hattori, T. (1996) A putative receptor protein kinase in Ipomoea trifida. Plant Cell Physiol. 37, 681-685.
Kyte, J. and Doolittle, R. F. (1982) A simple method for displaying the hydrophobic character of a protein. J. Mol. Biol. 157, 105-132.
Lange, J., Xie, Z.-P., Broughton, W. J., Vögeli-Lange, R., and Boller, T. (1999) A gene encoding a receptor-like protein kinase in the roots of common bean is differentially regulated in response to pathogens, symbionts and nodulation factors. Plant Sci. 142, 133-145.
McDonald, N. Q., Hendrickson, W. A. (1993) A structural superfamily of growth factors containing a cystine knot motif. Cell 73, 421-424.
Murashige, T., and Skoog, F. (1962) A revised medium for rapid growth and bioassays with tobacco tissue culture. Physiol. Plant. 15, 473-497.
Nasrallah, J. B., and Nasrallah, M. E. (1993) Pollen-Stigma signaling in the sporophytic self-incompatibility response. Plant Cell 5, 1325-1335.
Norman, C. Vidal, S., and Palva E. T. (1999) Oligogalacturonide-mediated induction of a gene involved in jasmonic acid synthesis in response to cell-wall-degrading enzymes of the plant pathogen Erwinia carotovora. Mol. Plant-Microbe Interact. 12, 640-644.
Norman-Setterblad, C., Vidal, S., and Palva, E. T. (2000) Interacting signal pathways control defense gene expression in Arabidopsis in response to cell wall-degrading enzymes from Erwinia carotovora. Mol. Plant-Microbe Interact. 13, 430-438.
Palva, T., K., Holmström, K.-O., Heino, P., and Palva, E. T. (1993) Induction of plant defense response by exoenzymes of Erwinia carotovora subsp. carotovora. Mol. Plant-Microbe Interact. 6, 190-196.
Pastuglia, M., Roby, D., Dumas, C., and Cock, M. (1997) Rapid induction by wounding and bacterial infection of a S gene family receptor-like kinase gene in Brassica oleracea. Plant Cell 9, 49-60.
Pérombelon, M. C. M., and Kelman, A. (1980) Ecology of the soft rot Erwinias. Annu. Rev. Phytopathol. 18,361-387.
Pirhonen, M., Heino, P., Helander, I., Harju, P., and Palva, E. T. (1988) Bacteriophage T4 resistant mutants of the plant pathogen Erwinia carotovora. Microb. Pathog. 4, 359-367.
Pirhonen, M., Saarilahti, H., Karlsson, M.-B., and Palva, E. T. (1991) Identification of pathogenicity determinants of Erwinia carotovora subsp. carotovora by transposon mutagenesis. Mol. Plant-Microbe Interact. 4, 276-283.
Satterlee, J. S. and Sussman, M. R. (1998) Unusual membrane-associated protein kinases in higher plants. J. Membr. Biol. 164, 205-213.
Sambrook, J. and Russell, D. W. (2001) Molecular cloning a laboratory manual. Cold Spring Harbor Laboratory Press, New York.
Singer, S. J. (1990) The structure and insertion of integral proteins in membranes. Annu. Rev. Cell Biol. 6, 247-296.
Smith, N. A., Singh, S. P., Wang, M.-B., Stoutjesdijk, P., Green, A. and Waterhouse, P. M. (2000) Total silencing by intron-spliced hairpin RNAs. Nature, 407, 319-320.
Song, W.-Y., Wang, G.-L., Chen, L.-L., Kim, H.-S., Pi, L.-Y., Holsten, T., Gardner, J., Wang, B., Zhai, W.-X., Zhu, L.-H., Fauquet, C., and Ronald, P. (1995) A receptor kinase-like protein encoded by the rice disease resistance gene, Xa21. Science 270, 1804-1806.
Stein, J. C., Howlett, B., Boyes, D., C., Nasrallah, M. E., and Nasrallah, J. B. (1991) Molecular cloning of a putative receptor protein kinase gene encoded at the self-incompatibility locus of Brassica olearacea. Proc. Natl. Acad. Sci. USA 88, 8816-8820.
Sun, P. D., and Davies, D. R. (1995) The cystine-knot growth-factor superfamily. Annu. Rev. Biomol. Struct. 24, 269-291.
Suzuki, A., Shioda, N., Maeda, T., Tada, M., Ueno, N. (1994) Cloning of an isoform of mouse TGF-β type II receptor gene. FEBS Lett. 355, 19-22.
Verwoerd, T. C., Dekker, M. M., and Hockema, A. (1989) A small-scale procedure for the rapid isolation of plant RNAs. Nucleic Acids Res. 17, 2362.
Vidal, S., Ponce de León, I., Denecke, J., and Palva, E. T. (1997) Salicylic acid and the plant pathogen Erwinia carotovora induce defense genes via antagonistic pathways. Plant J. 11, 115-123.
Vidal, S., Eriksson, A. R. B., Montesano, M., Denecke, J., and Palva, E. T. (1998) Cell wall-degrading enzymes from Erwinia carotovora cooperate in the salicylic acid-independent induction of plant defense response. Mol. Plant-Microbe Interact. 1, 23-32.
Walker, J. C. (1993) Receptor-like protein kinase genes of Arabidopsis thaliana. Plant J. 3, 451-456.
Walker, J. C. and Zhang, R. (1990) Relationship of a putative receptor protein kinase from maize to the S-locus glycoproteins of Brassica. Nature 345, 743-746.
Walker, J. C. (1994) Structure and function of the receptor-like protein kinase of higher plants. Plant Mol. Biol. 26, 1599-1609.
Wesley, S. V., Helliwell, C. A., Smith, N. A., Wang, M. B., Rouse, D. T., Liu. Q., Gooding, P. S., Singh, S. P., Abbott. D., Stoutjesdijk, P. A., Robinson, S. P., Gleave, A. P., Green, A. G. and Waterhouse P. M. (2001) Construct design for efficient, effective and high-throughput gene silencing in plants. Plant J. 27, 581-590.
Zambryski, P., Joost, H., Genetello, C., Leemans, J., Van Montagu, M. and Schell, J. (1983) Ti plasmid vector for the introduction of DNA into plant cells without alteration of their normal regeneration capacity. EMBO J. 2, 2143-2150.

What is claimed is:

1. An isolated nucleic acid sequence encoding potato receptor-like kinase, said nucleic acid sequence being at least 85% similar to nucleotide sequence according to SEQ ID NO:6 and comprising in extracellular domain one or more cystein repeats.

2. The isolated nucleic acid sequence according to claim 1, wherein the sequence is 91% similar to SEQ ID NO:6.

3. The isolated nucleic acid sequence according to claim 1, wherein the sequence is 99% similar to SEQ ID NO:6.

4. The isolated nucleic acid sequence according to claim 1, wherein the sequence is selected from a group consisting of SEQ ID NO:5, SEQ ID NO:6, SEQ ID NO:7 and SEQ ID NO:8.

5. The nucleic acid sequences according to claim 1 comprising a gene encoding an amino acid sequence isolated from potato plant under defined conditions and further being selected from the group consisting of SEQ ID NO:1, SEQ ID NO:2, SEQ ID NO:3 and SEQ ID NO:4.

6. The nucleic acid sequences according to claim 1, wherein expression of the potato receptor—like kinases is induced either by Erwinia carotovora or by response to elicitors.

7. Potato receptor-like kinase (PRK)-like gene products expressed by the nucleic acid sequences according to claim 1, wherein the PRK-like gene products are polypeptides comprising in their extracellular domains a conserved bi-modular patterns of one or more cysteine repeats.

8. The potato receptor-like kinase (PRK)-like gene products according to claim 7, further comprising polypeptides having amino acid sequences substantially homologous with an amino acid sequence selected from a group consisting of:
a. an amino acid sequence set forth in SEQ ID NO:1;
b. an amino acid sequence set forth in SEQ ID NO:2,
c. an amino acid sequence set forth in SEQ ID NO:3 and
d. an amino acid sequence set forth in SEQ ID NO:4.

9. A method for conferring resistance to pathogens in a plant, comprising a step of introducing into the plant a recombinant expression construct, said construct further comprising a plant promoter operably linked to a potato receptor-like kinase (PRK)-like nucleotide sequence of claim 1.

10. The method of claim 9, wherein potato receptor-like kinase (PRK)-like nucleotide sequence encodes a potato receptor-like kinase (PRK)-like gene product comprising polypeptide having amino acid sequence at least 85% similar to SEQ ID NO:2.

11. The method according to claim 10, wherein the amino acid sequence is at least 91% similar to SEQ ID NO:2.

12. The method according to claim 10, wherein the amino acid sequence is at least 99% similar to SEQ ID NO:2.

13. The method according to claim 10, wherein the amino acid sequence is selected from the group consisting of SEQ ID NO:1, SEQ ID NO:2; SEQ ID NO:3 and SEQ ID NO:4

14. The method of claim 9, wherein the plant is potato.

15. A DNA construct for cloning and/or transforming plants, wherein the DNA construct comprises nucleic acid sequences of claim 1 functionally combined with regulatory sequences.

16. The isolated nucleic acid sequence of claim 1, wherein the nucleic acid sequence further comprises at least one regulatory sequence for expressing said nucleic acid.

17. An expression vector comprising at least one nucleotide sequence of claim 1.

18. A host cell containing the DNA construct of claim 15.

19. A transgenic plant containing the DNA construct of claim 15.

20. The transgenic plant of claim 19 further over expressing potato receptor-like kinase (PRK)-like nucleotide sequence SEQ ID NO: 2.

21. A method for conferring resistance to pathogens in a plant, comprising a step of introducing into the plant a construct comprising an Arabidopsis receptor-like kinase (PRK)-like nucleotide sequence SEQ ID NO: 9 and at least one regulatory sequence for expressing said nucleotide sequence

22. The method of claim 21, wherein Arabidopsis receptor-like kinase (PRK)-like nucleotide sequence encodes a gene product of SEQ ID NO:14.
